(19) United States
(12) Patent Application Publication
(10) Pub. No.: US 2011/0021607 A1
(43) Pub. Date: Jan. 27, 2011

(54) Methods and Compositions Relating to Carcinoma Stem Cells

(76) Inventors: Michael Clarke, Palo Alto, CA (US); Yohei Shimono, Aichi (JP)

Correspondence Address: Stanford University Office of Technology Licensing;Bozicevic, Field & Francis LLP, 1900 University Avenue, Suite 200, East Palo Alto, CA, 94303, US

(21) Appl. No.: 12/865,090

(22) PCT Filed: Jan. 30, 2009
(86) PCT No.: PCT/US09/00593

§ 371 Date: Oct. 12, 2010

Related U.S. Application Data
(60) Provisional application No. 61/025,574, filed on Feb. 1, 2008.

(51) Int. Cl. A61K 31/7088; C12N 5/09; C12N 5/071; A61P 35/00

(52) U.S. Cl. 514/44 A; 435/6; 435/375

(57) ABSTRACT

MicroRNA markers of breast cancer stem cells (BCSC) are provided herein. The markers are polynucleotides that are differentially expressed in BCSC as compared to normal counterpart cells. Uses of the markers include use as targets for therapeutic intervention; as targets for drug development, and for diagnostic or prognostic methods relating to breast cancer and BCSC cell populations. BCSCs have the phenotype of having lower expression of certain miRNAs compared to normal breast epithelial cells, or to cancer cells that are not cancer stem cells.

### GOVERNMENT RIGHTS

This invention was made with Government support under contract CA104987 awarded by the NIH National Cancer Institute. The Government has certain rights in this invention.

### BACKGROUND OF THE INVENTION

Breast cancer is the most common malignancy in US women. Although therapies currently available can produce shrinkage in metastases, these effects are transient and the vast majority of people with stage 4 breast cancer succumb to it. Traditional modes of therapy, including radiation therapy, chemotherapy and hormonal therapy, have been useful but are limited by the emergence of treatment resistant cancer cells. New approaches are needed to detect and treat breast cancer.

Like many other types of solid tumors, the major cause of mortality is the spreading of the cancer from the site of origin to distant organs and tissues. This is a result of invasion of cancer cells from the initial tumor into the surrounding breast tissue as well as tissue lymphatic and blood vasculature. The invading cancer cells then form new tumors that eventually impair the function of critical organs to which the cancer has spread such as the liver, lung, or brain and eventually cause the death of the patient. Since the major cause of mortality from breast cancer is from dissemination of the cancer to other organs, one must either prevent the spread of tumor cells or eradicate distant tumors in order to improve survival.

A tumor can be viewed as an aberrant organ initiated by a tumorigenic cancer cell that acquired the capacity for indefinite proliferation through accumulated mutations. In this view of a tumor as an abnormal organ, the principles of normal stem cell biology can be applied to better understand how tumors develop and disseminate. Many observations suggest that analogies between normal stem cells and tumorigenic cells are appropriate. Both normal stem cells and tumorigenic cells have extensive proliferative potential and the ability to give rise to new (normal or abnormal) tissues. Tumorigenic cells can be thought of as cancer stem cells (CSC) that undergo an aberrant and poorly regulated process of organogenesis analogous to what normal stem cells do. Both tumors and normal tissues are composed of heterogeneous combinations of cells, with different phenotypic characteristics and different proliferative potentials.

It was found in acute myeloid leukaemia that only a small subset of cancer cells is responsible for the tumor-initiating potential and maintains the ability to self-renew. Because the differences in clonogenicity among the leukemia cells mirrored the differences in clonogenicity among normal hematopoietic cells, the clonogenic leukemic cells were described as leukemic stem cells. It has also been shown for solid cancers that the cells are phenotypically heterogeneous and that only a small proportion of cells are tumorigenic and can self-renew in vivo. Just as in the context of leukemic stem cells, these observations led to the hypothesis that only rare cancer stem cells exist in epithelial tumors.

Tumorigenic and non-tumorigenic populations of breast cancer cells can also be isolated based on their expression of cell surface markers. In many cases of breast cancer, only a small subpopulation of cells had the ability to form new tumors. Breast cancer tumors from many patients contain a subpopulation of cancer cells that can form tumors in immunodeficient mice while the other cancer cells cannot. As few as 100 tumorigenic cancer cells are able to form tumors when injected into immunodeficient mice and the resultant tumors contained the phenotypically heterogeneous populations of tumorigenic and non-tumorigenic cancer cells found in the patient's original tumor.

Further evidence for the existence of CSC occurring in solid tumors has been found in central nervous system (CNS) malignancies. Using culture techniques similar to those used to culture normal neuronal stem cells it has been shown that neuronal CNS malignancies contain a small population of cancer cells that are clonogenic in vitro and initiate tumors in vivo, while the remaining cells in the tumor do not have these properties. Importantly, the principles of stem cell biology have great applicability in the understanding of the biology of breast cancer tumors.

The presence of cancer stem cells has profound implications for cancer therapy. At present, all of the phenotypically diverse cancer cells in a tumor are treated as though they have unlimited proliferative potential and can acquire the ability to metastasize. For many years, however, it has been recognized that small numbers of disseminated cancer cells can be detected at sites distant from primary tumors in patients that never manifest metastatic disease. One possibility is that most cancer cells lack the ability to form a new tumor such, that only the dissemination of rare cancer stem cells can lead to metastatic disease. Hence, the goal of therapy must be to identify and kill this cancer stem cell population.

Existing therapies have been developed largely against the bulk population of tumor cells, because the therapies are identified by their ability to shrink the tumor mass. However, because most cells within a cancer have limited proliferative potential, an ability to shrink a tumor mainly reflects an ability to kill these cells. Therapies that are more specifically directed against cancer stem cells may result in more durable responses and cures of metastatic tumors.

mRNAs are small noncoding regulatory RNAs that regulate the translation of mRNAs by inhibiting ribosome function, de-capping the 5′cap structure, deadenylating the polyA tail, and degradation of the target mRNA. mRNAs are able to regulate expression of hundreds of mRNAs simultaneously and thus control a variety of cell functions including cell proliferation, stem cell maintenance and differentiation. One of the best studied miRNAs, let-7 in Caenorhabditis elegans, was initially identified by genetic analysis of mutants with defects in developmental timing. Subsequently, Dicer1 was identified as a key enzyme of miRNA processing and function; Dicer1 null mutations result in embryonic lethality and depletion of stem cells. In addition, tissue specific deletion of Dicer affects self-renewal of embryonic stem cells, development of B lymphocyte lineage cells, and tissue morphogenesis. In the skin, miR-203 is critical for development. Deletion of DGCR8, another key enzyme for miRNA processing, also alters silencing of self-renewal genes in embryonic stem cell differentiation. These findings demonstrate that miRNAs are critical regulators of tissue maintenance and differentiation. Recent studies have shown that many of the common chromosomal amplifications and deletions seen in cancers contain miRNA coding sequences, and that some miRNAs function as oncogenes or tumor suppressor genes. For example, dysregulation of the miR-17-92 cluster can induce B-cell lymphoma and down-regulation of let-7 is associated with tumor progression and poor prognosis of lung cancer patients. Expression of let-7 also prevents tumor sphere formation of breast cell lines and inhibits tumorigenicity in an in vivo xenograft tumor assay.

The subject invention is related to detection and manipulation of microRNAs in cancer stem cells. The ability to prospectively identify an enriched population of stem cells enables the interrogation of these cells for clues to the molecular regulators of key stem cell functions.

Cancer stem cells are discussed in, for example, Pardal et al. (2003) Nat Rev Cancer 3, 895-902; Reya et al. (2001) Nature 414, 105-11; Bonnet & Dick (1997) Nat Med 3, 730-7; Al-Hajj et al. (2003) Proc Natl Acad Sci USA 100, 3983-8; Dontu et al. (2004) Breast Cancer Res 6, R605-15; Singh et al. (2004) Nature 432, 396-401.

### SUMMARY OF THE INVENTION

MicroRNA markers of breast cancer stem cells (BCSC) are provided herein. The markers are polynucleotides that are differentially expressed in BCSC as compared to normal counterpart cells and as compared to non-tumorigenic cells found in breast cancer. Uses of the markers include use as targets for therapeutic intervention; as targets for drug development, and for diagnostic or prognostic methods relating to breast cancer and BCSC cell populations.

In some embodiments of the invention, methods are provided for treating breast cancer, the method comprising providing microRNA activity, e.g. through introduction of an expression vector or direct provision of microRNA to BCSC. MicroRNAs of interest for upregulation are shown herein to be downregulated in BCSC, and include, without limitation, microRNAs in the 200c-141 cluster (miR200c, miR141); in the 200b-200a-429 cluster (miR200b, miR200a, miR429); and in the 182-96-183 cluster (miR182, miR96, miR183).

In other embodiments, methods of treating breast cancer are provided where microRNA expression is downregulated. MicroRNAs of interest for down-regulation include, without limitation, miR214; miR-127; miR142-3p; miR-199a; miR1-125b; miR-146b; miR199b, and miR-222.

In some embodiments of the invention, methods are provided for classification or clinical staging of cancer, where greater numbers of BCSCs are indicative of a more aggressive cancer phenotype. Staging is useful for prognosis and treatment. In some embodiments of the invention, a tumor sample is analyzed by histochemistry, including immunohistochemistry, in situ hybridization, and the like, for the presence of such cells having decreased expression of the miRNAs identified herein. The presence of such cells indicates the presence of BCSCs, and allows the definition of BCSC microdomains in the primary tumor, as well as cells in lymph node or distant metastases. Identifying BCSCs by phenotype unique to them provides a more specific target than conventional therapies. Further, an embodiment of the invention also provides a means of predicting disease progression, relapse, and development of drug resistance.

In another embodiment of the invention, the miRNAs or their targets may be used, for example, in a method of screening for a compound that increases the expression of such miRNAs or to decrease the expression of their protein targets in cancer stem cells. This involves combining the compound with a cell population expression with a low expression of the miRNAs, and then determining any modulatory effect resulting from the compound. This may include examination of the cells for activity or detection of certain protein targets, viability, toxicity, metabolic change, or an effect on cell function. Methods are also provided for administration of therapeutic agents that target cancer stem cells that are related to the functions of miRNA disclosed herein.

### BRIEF DESCRIPTION OF THE DRAWINGS

FIG. 1. Profile of Human Breast Cancer Stem Cell miRNA Expression (A) Breast cancer miRNA screen. The details of the screen used to identify the 37 miRNAs differentially expressed by the CD44⁺CD24^{-/low} lineage⁻ tumorigenic cancer cells (TG cells) and the remaining lineage− non-tumorigenic cancer cells (NTG cells) are shown schematically. (B) Expression profile of 37 miRNAs in tumorigenic human breast cancer cells. Flow cytometry was used to isolate TG cells and NTG cells from 11 human breast cancer samples (BC1 to BC11). The amount of miRNA expression (Ct value) in 100 sorted cancer cells was analyzed by multiplex quantitative real-time PCR. Numbers represent the difference of Ct values (ΔCt) obtained from TG cells and NTG cells. (C) A schematic representation of the three miRNA clusters down-regulated in tumorigenic human breast cancer cells. The miRNAs sharing the same seed sequence (from 2 to 7 base pairs) are marked by the same color. (D) mRNA expression in Tera-2 embryonal carcinoma cells as compared to human breast cancer cells. The intensity of the miRNA expression in 100 cells of Tera-2 cells was compared to the miRNA expression in 100 cells of human breast cancer TG and NTG cells (BC1-BC11) by multiplex quantitative real-time PCR. The Ct values obtained from the 11 sets of breast cancer TG and NTG cells were averaged. Numbers represent the difference of Ct values (ΔCt) obtained from Tera-2 cells, human breast cancer TG as compared to NTG cells.

FIG. 2. Profile of Down-regulated miRNAs Shared Between Normal and Malignant Mammary Stem Cells (A) Distribution of CD45⁻CD31⁻CD140a⁻Ter119⁻ mouse mammary cells according to their expression of CD24 and CD49f. MRU is a population enriched for mammary stem cells. MaCFCs are progenitors that do not regenerate mammary gland in vivo. (B) Expression of miRNAs in MRUs as compared to MaCFCs. The expression of the miRNAs down-regulated in tumorigenic human breast cancer cells was analyzed in MRUs and MaCFCs isolated by flow cytometry from normal mouse mammary fat pads. The level of miRNA expression in 100 MRUs and MaCFCs was measured by quantitative real-time PCR. The analysis was repeated twice by using the two sets of samples derived from independently isolated populations of MRUs and MaCFCs. Numbers represent the difference of Ct values obtained from MRUs and MaCFCs.

FIG. 3. MiR-200c Targets SOX2 (A) Schematic representation of the miR-200bc/429 target sequence within the 3′ UTR of SOX2. Two nucleotides (corresponding to nucleotide 6 and 8 of miR-200bc/429) were mutated in the 3′UTR of SOX2. The numbers indicate the position of the nucleotides in the reference wild type sequences (NM_{—}003106). (B) Activity of the luciferase gene linked to the 3′UTR of SOX2. The pGL3 firefly luciferase reporter plasmids with the wild type or mutated 3′ UTR sequences of SOX2 were transiently transfected into HEK293T cells along with a Renilla luciferase reporter for normalization. Luciferase activities were measured after 48 hours. The mean of the results from the cells transfected by pGL3 control vector was set as 100%. The data are mean and S.D. of separate transfections (n=4). (C) SOX2 protein expression by embryonal carcinoma cells. Tera-2 embryonal carcinoma cells infected by the indicated miRNA expressing lentivirus were collected by flow cytometry six days after infection. Lysates from 30,000 sorted Tera-2 cells infected with a control lentivirus or a lentivirus expressing the indicated miRNA were loaded in each lane and SOX2 expression was analyzed by Western blotting. Expression of β-actin was used as a control. (D) Differential expression of SOX2 protein in TG and NTG cancer cells isolated from a primary human breast cancer sample. A primary human breast cancer sample was dissociated and CD44⁺CD24^{-/low} lineage⁻ tumorigenic cancer cells and the remaining non-tumorigenic lineage− cancer cells were collected by flow cytometry. Lysates from 6,000 sorted cells were loaded in each lane and SOX2 expression was analyzed by Western blotting. Expression of β-actin was used as a control.

FIG. 4. Growth Suppression of Embryonal Carcinoma Cells by miR-200c and miR-183. (A) Images of miRNA-expressing embryonal carcinoma cells. Tera-2 cells infected with the indicated miRNA expressing lentivirus were collected by flow cytometry four days after infection. Tera-2 cells were cultured for 19 days and stained with Giemsa Wright staining solution. (B) MiR-200c and miR-183 enhance differentiation of embryonal carcinoma cells. Tera-2 cells infected and collected as described in (A) were stained with primary antibody against the early post-mitotic neuron marker, Tuj1 followed by Alexa-488 labeled secondary antibody. Cells were counterstained with DAPI. (C) MiR-200c and miR-183 inhibited the growth of embryonal carcinoma cells in vitro. 3000 Tera-2 cells expressing a control or indicated miRNA were collected as described in (A) and cultured in a 96-well plate. Total cell numbers were counted on day 7, 12 and 19. The result is the average and S.D. from three independent wells.

FIG. 5. Effect of miR-200c and miR-183 on Clonogenicity of MMTV-Wnt-1 Murine Breast Cancer Cells (A) The incidence of colony formation by MMTV-Wnt-1 breast cancer cells expressing miR-200c and miR-183. MMTV-Wnt-1 breast cancer cells were dissociated and lineage positive cells were depleted using flow cytometry. 15,000 breast cancer cells were infected by the indicated miRNA-expressing lentivirus and cultured on irradiated 3T3 feeder layer in a 24-well plate. After 6 days of incubation, the number of colonies with more than 10 GFP positive cells was counted. The result shows the average and S.D. from four independent wells. (B) Immunofluorescence images of colonies stained with antibodies against cytokeratin 14, 19, and 8/18. The GFP positive colonies were marked and stained with primary antibodies against cytokeratins followed by Alexa-488 and Alexa-594 labeled secondary antibodies. Cells were counterstained with DAPI.

FIG. 6. Tumor Growth of Embryonal Carcinoma Cells Expressing miR-200c and miR-183 in vivo (A) A representative tumor in a mouse injected with Tera-2 embryonal carcinoma cells. Tera-2 cells were infected by the indicated miRNA-expressing lentivirus and the GFP-expressing cells were collected using flow cytometry. 50,000 GFP⁺ Tera-2 cells infected with the indicated lentivirus were injected subcutaneously into immunodeficient NOD/SCID mice. Tumor growth was monitored for three months after injection. The expression of miRNAs by the infected Tera-2 cells was confirmed by real-time PCR analysis. (B) Tumor incidence of miRNA-expressing Tera-2 cells. Three out of three control lentivirus infected Tera-2 cells developed tumors after three months. No miR-200c or miR-183 expressing Tera-2 cells formed a tumor. The result is a summary of three independent tumor injection experiments.

FIG. 7. Effect of miRNA expression on mammary outgrowth.

### DETAILED DESCRIPTION OF THE EMBODIMENTS

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims. In this specification and the appended claims, the singular forms “a,” “an” and “the” include plural reference unless the context clearly dictates otherwise.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing the subject components of the invention that are described in the publications, which components might be used in connection with the presently described invention.

As summarized above, the subject invention is directed to methods of classification of cancers, as well as reagents and kits for use in practicing the subject methods. The methods may also determine an appropriate level of treatment for a particular cancer.

Methods are also provided for optimizing therapy, by first classification, and based on that information, selecting the appropriate therapy, dose, treatment modality, etc. which optimizes the differential between delivery of an anti-proliferative treatment to the undesirable target cells, while minimizing undesirable toxicity. The treatment is optimized by selection for a treatment that minimizes undesirable toxicity, while providing for effective anti-proliferative activity.

The invention finds use in the prevention, treatment, detection or research of carcinomas, e.g. breast carcinomas. Carcinomas are cancers comprising neoplastic cells of epithelial origin. Epithelial cells cover the external surface of the body, line the internal cavities, and form the lining of glandular tissues. In adults, carcinomas are the most common forms of cancer. Carcinomas include the a variety of adenocarcinomas, for example in prostate, lung, etc.; adernocartical carcinoma; hepatocellular carcinoma; renal cell carcinoma, ovarian carcinoma, carcinoma in situ, ductal carcinoma, carcinoma of the breast, basal cell carcinoma; squamous cell carcinoma; transitional cell carcinoma; colon carcinoma; nasopharyngeal carcinoma; multilocular cystic renal cell carcinoma; oat cell carcinoma, large cell lung carcinoma; small cell lung carcinoma; etc. Carcinomas may be found in prostrate, pancreas, colon, brain (usually as secondary metastases), lung, breast, skin, etc.

Certain phenotypic attributes of carcinoma stem cells have been described in the art, and may include markers such as CD44, CD133, CD24, CD49f; ESA; CD166; and lineage panels. Examples of specific marker combinations and phenotypes are described, for example, by Al-Hajj et al. (2003) Prospective identification of tumorigenic breast cancer cells. Proc Natl Acad Sci USA 100, 3983-8; Singh et al. (2004) Identification of human brain tumour initiating cells. Nature 432, 396-401; Dalerba et al. (2007) Phenotypic characterization of human colorectal cancer stem cells. Proc Natl Acad Sci USA 104, 10158-63; O'Brien et al. (2006) A human colon cancer cell capable of initiating tumour growth in immunodeficient mice. Nature; Prince et al. (2007) Identification of a subpopulation of cells with cancer stem cell properties in head and neck squamous cell carcinoma. Proc Natl Acad Sci USA, each of which is herein specifically incorporated by reference for the teachings of cancer stem cell marker phenotypes. In some embodiments of the invention such phenotyping is used in conjunction with the detection of microRNA species.

The term “cancer stem cells,” as defined herein, refers to a subpopulation of tumorigenic cancer cells with both self-renewal and differentiation capacity. These tumorigenic cells are responsible for tumor maintenance and also give rise to large numbers of abnormally differentiating progeny that are not tumorigenic. These cells were able to initiate tumor growth at a dose of from about 10² cells, about 5×10² cells, about 10³ cells, providing at least a 100 fold increase in tumor initiating potential compared to the CD44 negative tumor cells. CD44 positive staining at the cell membrane allows the definition of cancer stem cell microdomains in a primary tumor. The presence of such microdomains is useful in diagnosis of cell carcinoma in primary and metastatic sites, where increased numbers of such microdomains is indicative of tumors with a greater capacity for tumorigenesis. These cells form tumors in vivo; self-renew to generate tumorigenic progeny; give rise to abnormally differentiated, nontumorigenic progeny, and differentially express at least one stem cell-associated gene. A population of cancer stem cells may be enriched by selecting for cells that express the cell surface marker CD44. In the case of breast cancer, cells within the CD44+CD24^{-/low}Lineage⁻ population possess the unique properties of cancer stem cells in functional assays for cancer stem cell self-renewal and differentiation, and form unique histological microdomains that can aid in cancer diagnosis. This population has higher tumorigenic capacity when compared with other cancer cell subsets, e.g. as shown by the use of murine xenograft assays. The lineage panel will usually include reagents specific for markers of normal leukocytes, fibroblasts, endothelial, mesothelial cells, etc.

“MicroRNAs (miRNAs),” as referred herein, are an abundant class of non-coding RNAs that are believed to be important in many biological processes through regulation of gene expression. They are single stranded RNA molecules that range in length from about 20 to about 25 nt, such as from about 21 to about 24 nt, e.g., 22 or 23 nt. These noncoding RNAs that can play important roles in development by targeting the messages of protein-coding genes for cleavage or repression of productive translation. Humans have between 200 and 255 genes that encode miRNAs, an abundance corresponding to almost 1% of the protein-coding genes. miRNAs are single stranded RNA molecules that range in length from about 20 to about 25 nt, such as from about 21 to about 24 nt, e.g., 22 or 23 nt.

In some embodiments, the miRNA markers are differentially expressed as a level reduced relative to a comparable non-tumorigenic cell, and may be reduced at least 2×, at least 3×, at least 4×, at least 10× or more.

The present invention provides methods of using the markers described herein in diagnosis of cancer, classification and treatment of cancers, particularly carcinomas. The methods are useful for characterizing CSC, facilitating diagnosis and the severity of the cancer (e.g., tumor grade, tumor burden, and the like) in a subject, facilitating a determination of the prognosis of a subject, and assessing the responsiveness of the subject to therapy. The detection methods of the invention can be conducted in vitro or in vivo, on isolated cells, or in whole tissues or a bodily fluid, e.g., blood, lymph node biopsy samples, and the like.

As used herein, the terms “a miRNA that is differentially expressed in a cancer stem cell,” and “a polynucleotide that is differentially expressed in a cancer stem cell”, are used interchangeably herein, and generally refer to a polynucleotide that represents or corresponds to a miRNA that is differentially expressed in a cancer stem cell when compared with a cell of the same cell type that is not tumorigenic, e.g., mRNA is found at levels at least about 25%, at least about 50% to about 75%, at least about 90%, at least about 1.5-fold, at least about 2-fold, at least about 3-fold, at least about 5-fold, at least about 10-fold, or at least about 50-fold or more, different.

A subject miRNA may be “identified” by a polynucleotide if the polynucleotide corresponds to or represents the miRNA, where an “identifying sequence” is a minimal fragment of a sequence of contiguous nucleotides that uniquely identifies or defines a polynucleotide sequence or its complement.

The term “biological sample” encompasses a variety of sample types obtained from an organism and can be used in a diagnostic or monitoring assay. The term encompasses blood and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain components. The term encompasses a clinical sample, and also includes cells in cell culture, cell supernatants, cell lysates, serum, plasma, biological fluids, and tissue samples.

Clinical samples for use in the methods of the invention may be obtained from a variety of sources, particularly biopsy samples, although in some instances samples such as blood, bone marrow, lymph, cerebrospinal fluid, synovial fluid, and the like may be used. Such samples can be separated by centrifugation, elutriation, density gradient separation, apheresis, affinity selection, panning, FACS, centrifugation with Hypaque, etc. prior to analysis. Once a sample is obtained, it can be used directly, frozen, or maintained in appropriate culture medium for short periods of time. Various media can be employed to maintain cells. The samples may be obtained by any convenient procedure, such as the drawing of blood, venipuncture, biopsy, or the like. Usually a sample will comprise at least about 10² cells, more usually at least about 10³ cells, and preferable 10⁴, 10⁵ or more cells. Typically the samples will be from human patients, although animal models may find use, e.g. equine, bovine, porcine, canine, feline, rodent, e.g. mice, rats, hamster, primate, etc.

An appropriate solution may be used for dispersion or suspension of the cell sample. Such solution will generally be a balanced salt solution, e.g. normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc.

Analysis of cell staining may use conventional methods. Techniques providing accurate enumeration include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g. propidium iodide).

Of particular interest is the use of antibodies as affinity reagents. Conveniently, these antibodies are conjugated with a label for use in separation. Labels include magnetic beads, which allow for direct separation, biotin, which can be removed with avidin or streptavidin bound to a support, fluorochromes, which can be used with a fluorescence activated cell sorter, or the like, to allow for ease of separation of the particular cell type. Fluorochromes that find use include phycobiliproteins, e.g. phycoerythrin and allophycocyanins, fluorescein and Texas red. Frequently each antibody is labeled with a different fluorochrome, to permit independent sorting for each marker.

The antibodies are added to a suspension of cells, and incubated for a period of time sufficient to bind the available cell surface antigens. The incubation will usually be at least about 5 minutes and usually less than about 30 minutes. It is desirable to have a sufficient concentration of antibodies in the reaction mixture, such that the efficiency of the separation is not limited by lack of antibody. The appropriate concentration is determined by titration. The medium in which the cells are separated will be any medium that maintains the viability of the cells. A preferred medium is phosphate buffered saline containing from 0.1 to 0.5% BSA. Various media are commercially available and may be used according to the nature of the cells, including Dulbecco's Modified Eagle Medium (dMEM), Hank's Basic Salt Solution (HBSS), Dulbecco's phosphate buffered saline (dPBS), RPMI, Iscove's medium, PBS with 5 mM EDTA, etc., frequently supplemented with fetal calf serum, BSA, HSA, etc. The labeled cells may then be quantitated as to the expression of cell surface markers as previously described.

“Diagnosis” as used herein generally includes determination of a subject's susceptibility to a disease or disorder, determination as to whether a subject is presently affected by a disease or disorder, prognosis of a subject affected by a disease or disorder (e.g., identification of cancerous states, stages of cancer, or responsiveness of cancer to therapy), and use of therametrics (e.g., monitoring a subject's condition to provide information as to the effect or efficacy of therapy).

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

The terms “individual,” “subject,” “host,” and “patient,” used interchangeably herein and refer to any mammalian subject for whom diagnosis, treatment, or therapy is desired, particularly humans:

A “host cell”, as used herein, refers to a microorganism or a eukaryotic cell or cell line cultured as a unicellular entity which can be, or has been, used as a recipient for a recombinant vector or other transfer polynucleotides, and include the progeny of the original cell which has been transfected. It is understood that the progeny of a single cell may not necessarily be completely identical in morphology or in genomic or total DNA complement as the original parent, due to natural, accidental, or deliberate mutation.

“Therapeutic target” refers to a gene or gene product that, upon modulation of its activity (e.g., by modulation of expression, biological activity, and the like), can provide for modulation of the cancerous phenotype. As used throughout, “modulation” is meant to refer to an increase or a decrease in the indicated phenomenon (e.g., modulation of a biological activity refers to an increase in a biological activity or a decrease in a biological activity).

### Breast Cell Carcinomas

Breast cancer is the most common malignancy in US women, affecting one in eight women during their lives. Risks for developing breast cancer are increased in certain cases, such as having a genetic predisposition by carrying the mutated BRCA1 or BRCA2 gene. “Breast cancer carcinoma,” as referred to herein, refers to epithelial tumors that develop from cells lining ducts or lobules. They are also often glandular in origin. Cancers are divided into carcinoma in situ and invasive cancer.

Carcinoma in situ is a proliferation of cancer cells within ducts or lobules and without invasion of stromal tissue. However, carcinoma in situ may also become invasive. Breast cancer invades locally and spreads initially through the regional lymph nodes, bloodstream, or both. Metastatic breast cancer may affect almost any organ in the body-most commonly, lungs, liver, bone, brain, and skin.

Symptoms of a possible breast malignancy include fibrotic changes, presence of lumps, and unusual discharge. If such symptom arises, testing is required to differentiate benign lesions from cancer. When advance cancer is suspected, a biopsy is usually performed first. Biopsy can be needle or incisional biopsy or, if the tumor is small, excisional biopsy.

For most patients, primary treatment is surgery, often with radiation therapy. Chemotherapy, hormone therapy, or both may also be used, depending on tumor and patient characteristics. For inflammatory or advanced breast cancer, primary treatment is systemic therapy, which, for inflammatory breast cancer, is followed by surgery and radiation therapy; surgery is usually not helpful for advanced cancer.

For patients with invasive cancer, chemotherapy or hormone therapy is usually begun soon after surgery and continued for months or years; these therapies delay or prevent recurrence in almost all patients and prolong survival in some. Combination chemotherapy regimens (eg, cyclophosphamide, methotrexate, 5-fluorouracil; doxorubicin plus cyclophosphamide) are often more effective than a single drug.

When cancer has metastasized, treatment increases median survival by only 3 to 6 months, although relatively toxic therapies (eg, chemotherapy) may palliate symptoms and improve quality of life. Choice of therapy depends on the hormone-receptor status of the tumor, length of the disease-free interval (from diagnosis to manifestation of metastases), number of metastatic sites and organs affected, and patient's menopausal status. Most patients with symptomatic metastatic disease are treated with systemic hormone therapy or chemotherapy. Some cytotoxic drugs for treatment of metastatic breast cancer are capecitabine, doxorubincin (including its liposomal formulation), gemcitabine, and the taxanes (paclitaxel, docetaxel, and vinorelbine).

### MicroRNA Probes and Targets in Carcinoma Stem Cells

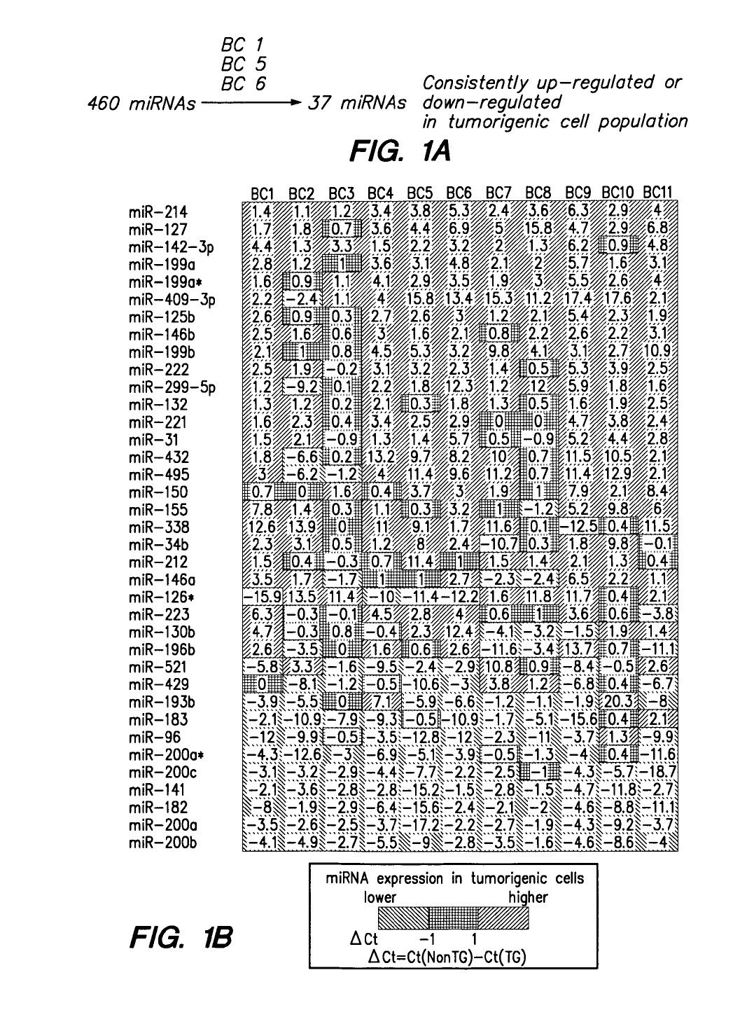

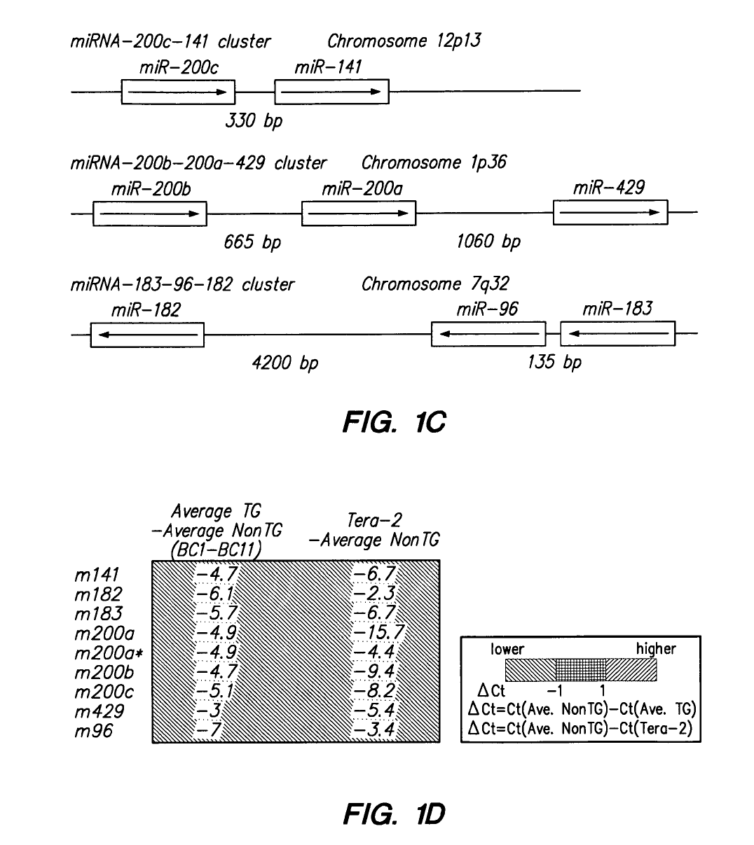

In some embodiments, microRNAS (miRNAs) for use in the subject method of the invention include those that are differentially expressed in BCSC relative to non-tumorigenic cells. MicroRNAs play important roles in regulating essential functions in the cell by targeting the messages of protein-coding genes for cleavage or repression of productive translation. In certain embodiments, the miRNA of interests presented are usually downregulated in BCSC. The nucleotide sequences of a subset of human miRNAs of interest are provided in Table 1. Other sequences of interest are listed in FIG. 1B, which miRNAs include miR-214; miR-127; miR-142-3p; miR-199a; miR-409-3p; miR-125b; miR-146b; miR-199b; miR-222; miR-299-5p; miR-132; miR-221; miR-31; miR-432; miR-495; miR-150; miR-155; miR-338; miR-34b; miR-212; miR-146a; miR-126; miR-223; miR-130b; miR-196b; miR-521; miR-429; miR-193b; miR-183; miR-96; miR-200a; miR-200c; miR-141; miR-182; miR-200a; miR-200b.

TABLE 1
a partial listing of MicroRNAs that are differentially expressed in
tumorigenic versus non-tumorigenic breast cancer cells.
Table 1 miRNA sequences

| | stem loop sequence | mature sequence |
|---|---|---|
| miR-200a | SEQ ID NO: 1 | SEQ ID NO: 2 |
| | ccgggccccugugagcaucuuaccggacagugcuggauuuccca | uaacacugucugguaacgaugu |
| | gcuugacucuaacacugucugguaacgauguucaaaggugaccc | |
| | gc | |
| miR-141 | SEQ ID NO: 3 | SEQ ID NO: 4 |
| | cggccggcccuggguccaucuuccaguacaguguuggauggucu | uaacacugucugguaaagaugg |
| | aauugugaagcuccuaacacugucugguaaagauggcucccggg | |
| | uggguuc | |
| miR-200b | SEQ ID NO: 5 | SEQ ID NO: 6 |
| | ccagcucgggcagccguggccaucuuacugggcagcauuggaug | uaauacugccugguaaugauga |
| | gagucaggucucuaauacugccugguaaugaugacggcggagcc | |
| | cugcacg | |
| miR-200c | SEQ ID NO: 6 | SEQ ID NO: 7 |
| | cccucgucuuacccagcaguguuugggugcgguugggagucucu | uaauacugccggguaaugaugga |
| | aauacugccggguaaugauggagg | |
| miR-429 | SEQ ID NO: 8 | SEQ ID NO: 9 |
| | cgccggccgaugggcgucuuaccagacaugguuagaccuggccc | uaauacugucugguaaaaccgu |
| | ucugucuaauacugucugguaaaaccguccauccgcugc | |
| miR-182 | SEQ ID NO: 10 | SEQ ID NO: 11 |
| | gagcugcuugccuccccccguuuuuggcaaugguagaacucaca | uuuggcaaugguagaacucacacu |
| | cuggugagguaacaggauccggugguucuagacuugccaacuau | |
| | ggggcgaggacucagccggcac | |
| miR-96 | SEQ ID NO: 12 | SEQ ID NO: 13 |
| | uggccgauuuuggcacuagcacauuuuugcuugugucucuccgc | uuuggcacuagcacauuuuugcu |
| | ucugagcaaucaugugcagugccaauaugggaaa | |
| miR-183 | SEQ ID NO: 14 | SEQ ID NO: 15 |
| | ccgcagagugugacuccuguucuguguauggcacugguagaauu | uauggcacugguagaauucacu |
| | cacugugaacagucucagucagugaauuaccgaagggccauaaa | |
| | cagagcagagacagauccacga | |

The miRNAs include those that are not identical in sequence to the disclosed nucleic acids, and variants thereof. Variant sequences can include nucleotide substitutions, additions or deletions.

In some embodiments, target proteins whose expression and regulation are affected by the downregulation of the miRNA presented above are provided to be used in the subject methods. This group of proteins include anti-apoptotic proteins such as the BCL-2 family members, transcriptional regulators, proto-oncogenes, oncogenes, and other proteins involved in the process of self-renewal. Some of the target proteins are described in more detail below.

ZFHX1B is a transcriptional repressor involved in the TGFβ signaling pathway and in processes of epithelial to mesenchymal transition (EMT) via regulation of E-cadherin. ZFHX1B and miR-200b are regionally coexpressed in the adult mouse brain. Overexpression of miR-200b leads to repression of endogenous ZFHX1B, and inhibition of miR-200b relieves the repression of ZFHX1B. The activity of the E-cadherin promoter is found to be regulated by both miR-200b and miR-200c.

BCL-2 family members either facilitate pro- or anti-apoptotic processes. Among those that are involved in anti-apoptosis include Bcl-2, Bcl-XL, Bcl-w, Mcl-1 and A1. They are known to regulate apoptosis via the permeability of the mitochondria membrane. High level expression of this protein family is implicated in carcinogenesis and the self-renewal of normal stem cells.

Another group of proteins that are targets of the miRNAs disclosed within is the polycomb-group proteins. Proteins belonging to this family can remodel chromatin such that transcription factors cannot bind to promoter sequences in DNA. The polycomb family proteins regulate critical events as cells undergo either renewal or senescence. One such family member is BMI1, whose mRNA target sequence is highly conserved across species. BMI1 is found to be downregulated in non-tumorigenic cancer cells.

Other targets of miRNA include MYB proto-oncogenes family members, expressed in hemopoietic cell lines and tissues where they are thought to be associated with the regulation of proliferation and differentiation.

Myc-family proteins are implicated in tumorigenesis and stem cell gene regulations (e.g. NMYC). Insulin-like growth factor binding proteins, such as IGFBP1, are also found to be linked to certain cancers and may also be regulated by miRNAs.

Another family of proteins that may be regulated by miRNAs is the Ras family of oncogenes. Ras oncogenes modulate signal transduction and cellular proliferation. In many carcinoma cases, accumulation of K-ras proteins correlates with an underlying K-ras gene-mutation.

Forkhead box 01A (FOX01A) is one essential transcription factor involved in the early steps of cellular differentiation and cell fusion. It also plays an important role in stem cell maintenance.

Another target of miRNA regulation is the SRY-related HMG-box (SOX) family of transcription factors. This family is involved in the regulation of embryonic development and in the determination of cell fate. SOX2, a member of this family, acts as a transcriptional activator after forming a protein complex with other proteins. It also plays a role in cell repair and DNA recombination.

These polynucleotides, polypeptides and fragments thereof have uses that include, but are not limited to, diagnostic probes and primers as starting materials for probes and primers, as immunogens for antibodies useful in cancer diagnosis and therapy, and the like as discussed herein.

Nucleic acid compositions include fragments and primers, and are at least about 15 by in length, at least about 30 by in length, at least about 50 by in length, at least about 100 bp, at least about 200 by in length, at least about 300 by in length, at least about 500 by in length, at least about 800 by in length, at least about 1 kb in length, at least about 2.0 kb in length, at least about 3.0 kb in length, at least about 5 kb in length, at least about 10 kb in length, at least about 50 kb in length and are usually less than about 200 kb in length. In some embodiments, a fragment of a polynucleotide is the coding sequence of a polynucleotide. Also included are variants or degenerate variants of a sequence provided herein. In general, variants of a polynucleotide provided herein have a fragment of sequence identity that is greater than at least about 65%, greater than at least about 70%, greater than at least about 75%, greater than at least about 80%, greater than at least about 85%, or greater than at least about 90%, 95%, 96%, 97%, 98%, 99% or more (i.e. 100%) as compared to an identically sized fragment of a provided sequence. as determined by the Smith-Waterman homology search algorithm as implemented in MPSRCH program (Oxford Molecular). Nucleic acids having sequence similarity can be detected by hybridization under low stringency conditions, for example, at 50° C. and 10×SSC (0.9 M saline/0.09 M sodium citrate) and remain bound when subjected to washing at 55° C. in 1×SSC. Sequence identity can be determined by hybridization under high stringency conditions, for example, at 50° C. or higher and 0.1×SSC (9 mM saline/0.9 mM sodium citrate). Hybridization methods and conditions are well known in the art, see, e.g., U.S. Pat. No. 5,707,829. Nucleic acids that are substantially identical to the provided polynucleotide sequences, e.g. allelic variants, genetically altered versions of the gene, etc., bind to the provided polynucleotide sequences under stringent hybridization conditions.

Probes specific to the miRNAs described herein can be generated using the polynucleotide sequences disclosed herein. The probes are usually a fragment of a polynucleotide sequences provided herein. The probes can be synthesized chemically or can be generated from longer polynucleotides using restriction enzymes. The probes can be labeled, for example, with a radioactive, biotinylated, or fluorescent tag. Preferably, probes are designed based upon an identifying sequence of any one of the polynucleotide sequences provided herein.

### Characterization of Carcinoma Stem Cells

In carcinomas, characterization of cancer stem cells allows for the development of new treatments that are specifically targeted against this critical population of cells, particularly their ability to self-renew, resulting in more effective therapies.

In human carcinomas, there is a subpopulation of tumorigenic cancer cells with both self-renewal and differentiation capacity. These tumorigenic cells are responsible for tumor maintenance, and also give rise to large numbers of abnormally differentiating progeny that are not tumorigenic, thus meeting the criteria of cancer stem cells. All tumorigenic potential was contained within the CD44+ Lineage− subpopulation of cancer cells. These cells were able to initiate tumor growth at a dose of from about 10³ cells, about 5×10³ cells, about 10⁴ cells, in comparison to while tumor suspension, which required a dose of around about 10⁶ cells to form a tumor, and a lack of tumor formation by CD44− Lineage− cells at much higher cell doses.

The breast cancer stem cells (BCSC) are identified by their phenotype with respect to particular markers, and/or by their functional phenotype. In some embodiments, the BCSC are identified and/or isolated by binding to the cell with reagents specific for the markers of interest, such as the presence or absence of a specific miRNA. The cells to be analyzed may initially be viable cells, or may be fixed or embedded cells. In one embodiment, real time PCR analysis is used to analyze miRNA expression. High level of a miRNA set forth in Table 1 may be indicative that the cells are non-tumorigenic or non-invasive, while low levels are indicative of CSC.

BCSC can be identified and/or characterized based on their expression levels of miRNAs set forth in Table 1. Low or undetectable levels of expression can indicate the presence of cancer stem cells. Normal breast epithelial cells or non-tumorigenic cancel cells can be used as a control when comparing expression levels of miRNAs or proteins.

In some embodiments, the reagents specific for the markers of interest are antibodies or polynucleotides, which may be directly or indirectly labeled. In certain cases, the antibodies are directed to specifically bind protein targets regulated by specific miRNAs disclosed, such as SOX2.

The protein or polynucleotide probes described previously can be used to, for example, determine the presence or absence of any one of the polynucleotide provided herein or variants thereof in a sample. These and other uses are described in more detail below.

Staining or hybridization with the various markers disclosed herein also allows the definition of cancer stem cell microdomains in the primary tumor. The presence of such microdomains is useful in diagnosis of squamous cell carcinoma in primary and metastatic sites, where increased numbers of such microdomains is indicative of tumors with a greater capacity for tumorigenesis.

### Differential Cell Analysis

The presence of BCSC in a patient sample can be indicative of the stage or grade of the carcinoma. Knowing the cancer cell subtypes and the location of BCSC can greatly aid in diagnosis and treatment. In addition, detection of BCSC can be used to monitor response to therapy and to aid in prognosis. Prognostic factors help determine treatment protocol and intensity; patients with strongly negative prognostic features are usually given more intense forms of therapy, because the potential benefits are thought to justify the increased treatment toxicity.

The presence of BCSC can be determined by quantifying cells having a phenotype of the stem cell as described herein. In addition to cell surface phenotyping, it may be useful to quantify cells in a sample that have a “stem cell” character, which may be determined by the expression profile of specific genes, expression of the provided miRNAs and target proteins, or by functional criteria, such as the ability to self-renew, to give rise to tumors in vivo, e.g. in a xenograft model, and the like.

One method that may be used is in situ hybridization for the miRNA species disclosed herein. Given the short length of the fragments of miRNA, locked nucleic acid (LNA) may be used as a probe, given its high melting temperature, in combination with known positive and negative controls for each miRNA species. Melting temperatures may vary across a wide range to determine the best probe labeling condition. Negative controls consisting of mismatched LNA probes may also be used, with 1, 2, 3 or 4 mismatches. Low or undetectable detection of the miRNA disclosed herein is one of the indicators of BCSC.

Another approach to determine the presence of miRNA species for diagnosis or prognosis is to combine RT-PCR with laser capture microdissection. Multiplex RT-PCR (reverse transcription-PCR) may be used to determine the presence of miRNA species in tissue samples.

Detection can also be accomplished by any known method, including, but not limited to, in situ hybridization, PCR (polymerase chain reaction), and “Northern” or RNA blotting, arrays, microarrays, etc, or combinations of such techniques, using a suitably labeled polynucleotide. A variety of labels and labeling methods for polynucleotides are known in the art and can be used in the assay methods of the invention.

The comparison of a differential progenitor analysis obtained from a patient sample, and a reference differential progenitor analysis is accomplished by the use of suitable deduction protocols, AI systems, statistical comparisons, etc. A comparison with a reference tissue analysis from normal cells, cells from similarly diseased tissue, and the like, can provide an indication of the disease staging. A database of reference tissue analyses can be compiled. The method of the invention provides detection of a predisposition to more aggressive tumor grow growth prior to onset of clinical symptoms, and therefore allow early therapeutic intervention, e.g. initiation of chemotherapy, increase of chemotherapy dose, changing selection of chemotherapeutic drug, and the like.

In certain embodiments, diagnosis and prognosis of breast cancer carcinoma may contain cell staining of tissue samples. In certain cases, cell staining may help delineate cancer cell subtypes within a tumor and locate invasive cancer cells. Analysis by cell staining may use conventional methods, as known in the art. Techniques providing accurate enumeration include confocal microscopy, fluorescence microscopy, fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g. propidium iodide).

Antibody reagents may be specific for proteins targeted by the miRNAs, or polynucleotide probes specific for the miRNAs themselves may also be used. In comparing to normal cells or non-tumorigenic controls, high expression of miRNA targets, or low expression of the miRNA indicates the presence of BCSC. Antibodies may be monoclonal or polyclonal, and may be produced by transgenic animals, immunized animals, immortalized human or animal B-cells, cells transfected with DNA vectors encoding the antibody or T cell receptor, etc. The details of the preparation of antibodies and their suitability for use as specific binding members are well-known to those skilled in the art.

Analysis may be performed based on in situ hybridization analysis, or antibody binding to tissue sections. Such analysis allows identification of histologically distinct cells within a tumor mass, and the identification of genes expressed in such cells. Sections for hybridization may comprise one or multiple solid tumor samples, e.g. using a tissue microarray (see, for example, West and van de Rijn (2006) Histopathology 48(1):22-31; and Montgomery et al. (2005) Appl Immunohistochem Mol. Morphol. 13(1):80-4). Tissue microarrays (TMAs) comprise multiple sections. A selected probe, e.g. antibody specific for a marker of interest, is labeled, and allowed to bind to the tissue section, using methods known in the art. The staining may be combined with other histochemical or immunohistochemical methods. The expression of selected genes in a stromal component of a tumor allows for characterization of the cells according to similarity to a stromal cell correlate of a soft tissue tumor.

### Screening Assays

In certain embodiments of the invention, the miRNAs or their targets may be used in a method of screening for a compound that can aid in research or drug development. In some embodiments, screening is performed to discover compounds or cellular factors that increase the expression of the provided miRNAs or that decrease the expression of their protein targets in cancer stem cells. This may involve combining a candidate agent with a cell population expressing low or zero amount of the miRNAs, e.g. a stem cell or cancer stem cell population, and then determining any modulatory effect resulting from the candidate. This may also include examination of the cells for activity or detection of certain protein targets, viability, toxicity, metabolic change, or an effect on cell function.

Of particular interest are screening assays for agents that are active on human cells. A wide variety of assays may be used for this purpose, including immunoassays for binding protein targets of miRNAs; determination of cell growth, differentiation and functional activity; production of factors; and the like. Specifically, assays may include analysis of expression of proteins identified herein as being regulated by miRNAs.

Screening may be performed using in vitro cultured cells, freshly isolated cells, a genetically altered cell or animal, purified miRNAs, purified protein regulated by miRNAs, and the like. In one embodiment, screening is performed to determine the activity of a candidate agent with respect to dampening the activity of miRNA target proteins. Such an agent may be tested by contacting the purified proteins with a candidate agent. Alternatively, a cell may be contacted with a candidate agent for regulation of transcription or translation of each of these proteins. In such assays, the miRNAs disclosed herein may serve as a positive control for coordinately regulating expression of these proteins. Compound screening identifies agents that modulate activity of the miRNA regulated proteins or the miRNAs themselves. Candidate compounds that increase the activity or expression of specific miRNAs may further the understanding of cancer biology and are important in the development of cancer therapeutics. Of particular interest are screening assays for agents that have a low toxicity for human cells.

The term “agent” as used herein describes any molecule, e.g. protein or pharmaceutical, with the capability of altering or mimicking the physiological function of a ischemia associated kinase corresponding to Ischemia associated genes. Generally a plurality of assay mixtures can be run in parallel with different agent concentrations to obtain a differential response to the various concentrations. Typically one of these concentrations serves as a negative control, i.e. at zero concentration or below the level of detection.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Test agents can be obtained from libraries, such as natural product libraries or combinatorial libraries, for example. A number of different types of combinatorial libraries and methods for preparing such libraries have been described, including for example, PCT publications WO 93/06121, WO 95/12608, WO 95/35503, WO 94/08051 and WO 95/30642, each of which is incorporated herein by reference.

A variety of other reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used. The mixture of components is added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 1 hour will be sufficient.

Certain screening methods involve screening for a compound that modulates the expression of proteins targeted by miRNAs, e.g. ZFHX1B, MYB proto-oncogene, and IGFBP1. Such methods generally involve conducting cell-based assays in which test compounds are contacted with one or more cells expressing the proteins and then detecting and a change in level of expression of the targeted proteins. Some assays are performed with cells enriched for tumorigenic or non-tumorigenic properties.

Expression can be detected in a number of different ways. The expression level of a gene in a cell can be determined by probing the mRNA expressed in a cell with a probe that specifically hybridizes with a transcript (or complementary nucleic acid derived therefrom) of the gene. Probing can be conducted by lysing the cells and conducting Northern blots or without lysing the cells using in situ-hybridization techniques. Alternatively, a protein can be detected using immunological methods in which a cell lysate is probe with antibodies that specifically bind to the protein.

Other cell-based assays are reporter assays. Certain of these assays are conducted with a heterologous nucleic acid construct that includes a promoter that is operably linked to a reporter gene that encodes a detectable product. A number of different reporter genes can be utilized. Some reporters are inherently detectable. An example of such a reporter is green fluorescent protein that emits fluorescence that can be detected with a fluorescence detector. Other reporters generate a detectable product. Often such reporters are enzymes. Exemplary enzyme reporters include, but are not limited to, β-glucuronidase, CAT (chloramphenicol acetyl transferase; Alton and Vapnek (1979) Nature 282:864-869), luciferase, β-galactosidase and alkaline phosphatase (Toh, et al. (1980) Eur. J. Biochem. 182:231-238; and Hall et al. (1983) J. Mol. Appl. Gen. 2:101).

In these assays, cells harboring the reporter construct are contacted with a test compound. A test compound that either activates a promoter by binding to it or triggers a cascade that produces the miRNA of interest causes expression of the detectable reporter. Certain other reporter assays are conducted with cells that harbor a heterologous construct that includes a transcriptional control element that activates expression. Here, too, an agent that binds to the transcriptional control element to activate expression of the reporter or that triggers the formation of an agent that binds to the transcriptional control element to activate reporter expression can be identified by the generation of signal associated with reporter expression.

The level of expression or activity can be compared to a baseline value. As indicated above, the baseline value can be a value for a control sample or a statistical value that is representative of a control population (e.g., healthy individuals). Expression levels can also be determined for cells that do not express the provided polynucleotide or proteins as a control. Such cells generally are otherwise substantially genetically the same as the test cells.

A variety of different types of cells can be utilized in the reporter assays. Eukaryotic cells may be used and can be any of the cells typically utilized in generating cells that harbor recombinant nucleic acid constructs. Exemplary eukaryotic cells include, but are not limited to, yeast, and various higher eukaryotic cells such as the COS, CHO and HeLa cell lines.

Various controls can be conducted to ensure that an observed activity is authentic including running parallel reactions with cells that lack the reporter construct or by not contacting a cell harboring the reporter construct with test compound. Compounds can also be further validated as described below.

Compounds and cellular substrates that are initially identified by any of the foregoing screening methods can be further tested to validate the apparent activity. The basic format of such methods involves administering the candidate identified during an initial screen to an animal that serves as a model for humans and then determining if a specific miRNA's or target protein's expression has changed. The animal models utilized in validation studies generally are mammals. Specific examples of suitable animals include, but are not limited to, primates, mice, and rats.

Certain methods are designed to test not only the ability of a lead candidate to alter activity in an animal model, but to provide protection against invasive cancer. In such methods, a lead compound is administered to the model animal (i.e., an animal, typically a mammal, other than a human). The animal is either predisposed to develop invasive carcinoma by its genetic makeup or by environmental factors or already has the carcinoma. Compounds or protein substrates able to achieve the desired effect of decreasing invasive cancer are good candidates for further study.

Active test agents identified by the screening methods described herein can serve as lead compounds for the synthesis of analog compounds. Typically, the analog compounds are synthesized to have an electronic configuration and a molecular conformation similar to that of the lead compound. Identification of analog compounds can be performed through use of techniques such as self-consistent field (SCF) analysis, configuration interaction (CI) analysis, and normal mode dynamics analysis. Computer programs for implementing these techniques are available. See, e.g., Rein et al., (1989) Computer-Assisted Modeling of Receptor-Ligand Interactions (Alan Liss, New York).

### Treatment of Cancer

The invention further provides methods for reducing growth of cancer cells. The method provides for decreasing the number of cancer cells bearing a specific marker or combination of markers, as provided herein, decreasing expression of a gene that is differentially expressed in a cancer cell, altering the level of miRNA expression, or decreasing the level of and/or decreasing an activity of a cancer-associated polypeptide. The method further includes introducing polynucleotides or polypeptides that would result in the effect of decreasing cancer growth. For example, a genetic construct encoding a miRNA set forth in Table 1 can be introduced into cancer stem cells to increase the miRNA level in the cell.

The term miRNA may refer to any of the provided sequences, usually in reference to the provided mature sequences. Included in the scope of the term “microRNA” is included synthetic molecules with substantially the same activity as the native microRNA, e.g. synthetic oligonucleotides having altered chemistries, as are known in the art.

In practicing the subject methods, an effective amount of a miR agent specific for, without limitation, microRNAs in the 200c-141 cluster (miR200c, miR141); in the 200b-200a-429 cluster (miR200b, miR200a, miR429); and in the 182-96-183 cluster (miR182, miR96, miR183) is introduced into the target cell, where any convenient protocol for introducing the agent into the target cell may be employed. The target cell is usually a carcinoma, including breast carcinoma, and more particularly including breast carcinoma stem cells, for example cells having the phenotype of being CD44⁺CD24^{-/low} lineage⁻ cells.

The subject methods are used for prophylactic or therapeutic purposes. As used herein, the term “treating” is used to refer to both prevention of disease, and treatment of pre-existing conditions. For example, the prevention of autoimmune disease may be accomplished by administration of the agent prior to development of overt disease. The treatment of ongoing disease, where the treatment stabilizes or improves the clinical symptoms of the patient, is of particular interest.

As is known in the art, miRNAs are single stranded RNA molecules that range in length from about 20 to about 25 nt, such as from about 21 to about 24 nt, e.g., 22 or 23 nt. The target miR181a may or may not be completely complementary to the introduced miR181a agent. If not completely complementary, the miRNA and its corresponding target viral genome are at least substantially complementary, such that the amount of mismatches present over the length of the miRNA, (ranging from about 20 to about 25 nt) will not exceed about 8 nt, and will in certain embodiments not exceed about 6 or 5 nt, e.g., 4 nt, 3 nt, 2 nt or 1 nt.

The miRNA agent may increase or decrease the levels of the targeted miRNA in the targeted cell. Where the agent is an inhibitory agent, it inhibits the activity of the target miRNA by reducing the amount of miRNA present in the targeted cells, where the target cell may be present in vitro or in vivo. By “reducing the amount of” is meant that the level or quantity of the target miRNA in the target cell is reduced by at least about 2-fold, usually by at least about 5-fold, e.g., 10-fold, 15-fold, 20-fold, 50-fold, 100-fold or more, as compared to a control, i.e., an identical target cell not treated according to the subject methods.

Where the miRNA agent increases the activity of the targeted miRNA in a cell, the amount of miRNA is increased in the targeted cells, where the target cell may be present in vitro or in vivo. By “increasing the amount of” is meant that the level or quantity of the target miRNA in the target cell is increased by at least about 2-fold, usually by at least about 5-fold, e.g., 10-fold, 15-fold, 20-fold, 50-fold, 100-fold or more, as compared to a control, i.e., an identical target cell not treated according to the subject methods.

By miRNA inhibitory agent is meant an agent that inhibits the activity of the target miRNA. The inhibitory agent may inhibit the activity of the target miRNA by a variety of different mechanisms. In certain embodiments, the inhibitory agent is one that binds to the target miRNA and, in doing so, inhibits its activity. Representative miRNA inhibitory agents include, but are not limited to: antisense oligonucleotides, and the like. Other agents of interest include, but are not limited to: Naturally occurring or synthetic small molecule compounds of interest, which include numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Such molecules may be identified, among other ways, by employing appropriate screening protocols.

The antisense reagent may be antisense oligonucleotides (ODN), particularly synthetic ODN having chemical modifications from native nucleic acids, or nucleic acid constructs that express such antisense molecules as RNA. The antisense sequence is complementary to the targeted miRNA, and inhibits its expression. One or a combination of antisense molecules may be administered, where a combination may comprise multiple different sequences.

Antisense molecules may be produced by expression of all or a part of the target miRNA sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 25, usually not more than about 23-22 nucleotides in length, where the length is governed by efficiency of inhibition, specificity, including absence of cross-reactivity, and the like.

Antisense oligonucleotides may be chemically synthesized by methods known in the art (see Wagner et al. (1993) supra. and Milligan et al., supra.) Preferred oligonucleotides are chemically modified from the native phosphodiester structure, in order to increase their intracellular stability and binding affinity. A number of such modifications have been described in the literature that alter the chemistry of the backbone, sugars or heterocyclic bases.

Among useful changes in the backbone chemistry are phosphorothioates;

phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH2-5′-O-phosphonate and 3′-NH-5′-O-phosphoroamidate. Peptide nucleic acids replace the entire ribose phosphodiester backbone with a peptide linkage. Sugar modifications are also used to enhance stability and affinity. The alpha.-anomer of deoxyribose may be used, where the base is inverted with respect to the natural .beta.-anomer. The 2′-OH of the ribose sugar may be altered to form 2′-O-methyl or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

Anti-sense molecules of interest include antagomir RNAs, e.g. as described by Krutzfeldt et al., supra., herein specifically incorporated by reference. Small interfering double-stranded RNAs (siRNAs) engineered with certain ‘drug-like’ properties such as chemical modifications for stability and cholesterol conjugation for delivery have been shown to achieve therapeutic silencing of an endogenous gene in vivo. To develop a pharmacological approach for silencing miRNAs in vivo, chemically modified, cholesterol-conjugated single-stranded RNA analogues complementary to miRNAs were developed, termed ‘antagomirs’. Antagomir RNAs may be synthesized using standard solid phase oligonucleotide synthesis protocols. The RNAs are conjugated to cholesterol, and may further have a phosphorothioate backbone at one or more positions.

Also of interest in certain embodiments are RNAi agents. In representative embodiments, the RNAi agent targets the precursor molecule of the microRNA, known as pre-microRNA molecule. By RNAi agent is meant an agent that modulates expression of microRNA by a RNA interference mechanism. The RNAi agents employed in one embodiment of the subject invention are small ribonucleic acid molecules (also referred to herein as interfering ribonucleic acids), i.e., oligoribonucleotides, that are present in duplex structures, e.g., two distinct oligoribonucleotides hybridized to each other or a single ribooligonucleotide that assumes a small hairpin formation to produce a duplex structure. By oligoribonucleotide is meant a ribonucleic acid that does not exceed about 100 nt in length, and typically does not exceed about 75 nt length, where the length in certain embodiments is less than about 70 nt. Where the RNA agent is a duplex structure of two distinct ribonucleic acids hybridized to each other, e.g., an siRNA, the length of the duplex structure typically ranges from about 15 to 30 bp, usually from about 15 to 29 bp, where lengths between about 20 and 29 bps, e.g., 21 bp, 22 bp, are of particular interest in certain embodiments. Where the RNA agent is a duplex structure of a single ribonucleic acid that is present in a hairpin formation, i.e., a shRNA, the length of the hybridized portion of the hairpin is typically the same as that provided above for the siRNA type of agent or longer by 4-8 nucleotides. The weight of the RNAi agents of this embodiment typically ranges from about 5,000 daltons to about 35,000 daltons, and in many embodiments is at least about 10,000 daltons and less than about 27,500 daltons, often less than about 25,000 daltons.

Where it is desirable to increase miRNA expression in a cell, e.g. to induce differentiation, an agent may be a microRNA itself, including any of the modified oligonucleotides described above with respect to antisense, e.g. cholesterol conjugates, phosphorothioates linkages, and the like. Alternatively, a vector that expresses a miRNA, including the pre-miRNA (hairpin) sequence relevant to the targeted organism, may be utilized.

Expression vectors may be used to introduce the target gene into a cell. Such vectors generally have convenient restriction sites located near the promoter sequence to provide for the insertion of nucleic acid sequences. Transcription cassettes may be prepared comprising a transcription initiation region, the target gene or fragment thereof, and a transcriptional termination region. The transcription cassettes may be introduced into a variety of vectors, e.g. plasmid; retrovirus, e.g. lentivirus; adenovirus; and the like, where the vectors are able to transiently or stably be maintained in the cells, usually for a period of at least about one day, more usually for a period of at least about several days to several weeks.

The expression cassette will generally employ an exogenous transcriptional initiation region, i.e. a promoter other than the promoter which is associated with the T cell receptor in the normally occurring chromosome. The promoter is functional in host cells, particularly host cells targeted by the cassette. The promoter may be introduced by recombinant methods in vitro, or as the result of homologous integration of the sequence by a suitable host cell. The promoter is operably linked to the coding sequence of the autoantigen to produce a translatable mRNA transcript. Expression vectors conveniently will have restriction sites located near the promoter sequence to facilitate the insertion of autoantigen sequences.

Expression cassettes are prepared comprising a transcription initiation region, which may be constitutive or inducible, the gene encoding the autoantigen sequence, and a transcriptional termination region. The expression cassettes may be introduced into a variety of vectors. Promoters of interest may be inducible or constitutive, usually constitutive, and will provide for high levels of transcription in the vaccine recipient cells. The promoter may be active only in the recipient cell type, or may be broadly active in many different cell types. Many strong promoters for mammalian cells are known in the art, including the .beta.-actin promoter, SV40 early and late promoters, immunoglobulin promoter, human cytomegalovirus promoter, retroviral LTRs, etc. The promoters may or may not be associated with enhancers, where the enhancers may be naturally associated with the particular promoter or associated with a different promoter.

A termination region is provided 3′ to the coding region, where the termination region may be naturally associated with the variable region domain or may be derived from a different source. A wide variety of termination regions may be employed without adversely affecting expression. The various manipulations may be carried out in vitro or may be performed in an appropriate host, e.g. E. coli. After each manipulation, the resulting construct may be cloned, the vector isolated, and the DNA screened or sequenced to ensure the correctness of the construct. The sequence may be screened by restriction analysis, sequencing, or the like.

As indicated above, the miRNA agent can be introduced into the target cell(s) using any convenient protocol, where the protocol will vary depending on whether the target cells are in vitro or in vivo. A number of options can be utilized to deliver the dsRNA into a cell or population of cells such as in a cell culture, tissue, organ or embryo. For instance, RNA can be directly introduced intracellularly. Various physical methods are generally utilized in such instances, such as administration by microinjection (see, e.g., Zernicka-Goetz, et al. (1997) Development 124:1133-1137; and Wianny, et al. (1998) Chromosoma 107: 430-439). Other options for cellular delivery include permeabilizing the cell membrane and electroporation in the presence of the dsRNA, liposome-mediated transfection, or transfection using chemicals such as calcium phosphate. A number of established gene therapy techniques can also be utilized to introduce the dsRNA into a cell. By introducing a viral construct within a viral particle, for instance, one can achieve efficient introduction of an expression construct into the cell and transcription of the RNA encoded by the construct.

For example, the inhibitory agent can be fed directly to, injected into, the host organism containing the target gene. The agent may be directly introduced into the cell (i.e., intracellularly); or introduced extracellularly into a cavity, interstitial space, into the circulation of an organism, introduced orally, etc. Methods for oral introduction include direct mixing of RNA with food of the organism. Physical methods of introducing nucleic acids include injection directly into the cell or extracellular injection into the organism of an RNA solution. The agent may be introduced in an amount which allows delivery of at least one copy per cell. Higher doses (e.g., at least 5, 10, 100, 500 or 1000 copies per cell) of the agent may yield more effective inhibition; lower doses may also be useful for specific applications.

When liposomes are utilized, substrates that bind to a cell-surface membrane protein associated with endocytosis can be attached to the liposome to target the liposome to T cells and to facilitate uptake. Examples of proteins that can be attached include capsid proteins or fragments thereof that bind to T cells, antibodies that specifically bind to cell-surface proteins on T cells that undergo internalization in cycling and proteins that target intracellular localizations within T cells. Gene marking and gene therapy protocols are reviewed by Anderson et al. (1992) Science 256:808-813.

In certain embodiments, a hydrodynamic nucleic acid administration protocol is employed. Where the agent is a ribonucleic acid, the hydrodynamic ribonucleic acid administration protocol described in detail below is of particular interest. Where the agent is a deoxyribonucleic acid, the hydrodynamic deoxyribonucleic acid administration protocols described in Chang et al., J. Virol. (2001) 75:3469-3473; Liu et al., Gene Ther. (1999) 6:1258-1266; Wolff et al., Science (1990) 247: 1465-1468; Zhang et al., Hum. Gene Ther. (1999) 10:1735-1737: and Zhang et al., Gene Ther. (1999) 7:1344-1349; are of interest.

Additional nucleic acid delivery protocols of interest include, but are not limited to: those described in U.S. patents of interest include U.S. Pat. Nos. 5,985,847 and 5,922,687 (the disclosures of which are herein incorporated by reference); WO/11092; Acsadi et al., New Biol. (1991) 3:71-81; Hickman et al., Hum. Gen. Ther. (1994) 5:1477-1483; and Wolff et al., Science (1990) 247: 1465-1468; etc.

Depending on the nature of the agent, the active agent(s) may be administered to the host using any convenient means capable of resulting in the desired modulation of miRNA in the target cell. Thus, the agent can be incorporated into a variety of formulations for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols. As such, administration of the agents can be achieved in various ways, including oral, buccal, rectal, parenteral, intraperitoneal, intradermal, transdermal, intracheal, etc., administration.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Those of skill in the art will readily appreciate that dose levels can vary as a function of the specific compound, the nature of the delivery vehicle, and the like. Preferred dosages for a given compound are readily determinable by those of skill in the art by a variety of means. Introduction of an effective amount of a miRNA agent into a mammalian cell as described above results in a modulation of target gene(s) expression, resulting in a modification of the carcinoma tumorigenic activity, thus providing a means of treating a cancer with a method that targets cancer stem cells.

“Reducing growth of cancer cells” includes, but is not limited to, reducing proliferation of cancer cells, and reducing the incidence of a non-cancerous cell becoming a cancerous cell. Whether a reduction in cancer cell growth has been achieved can be readily determined using any known assay, including, but not limited to, [³H]-thymidine incorporation; counting cell number over a period of time; detecting and/or measuring a marker associated with BCSC, etc.

The present invention provides methods for treating cancer, generally comprising administering to an individual in need thereof a substance that reduces cancer cell growth, in an amount sufficient to reduce cancer cell growth and treat the cancer. Whether a substance, or a specific amount of the substance, is effective in treating cancer can be assessed using any of a variety of known diagnostic assays for cancer, including, but not limited to biopsy, contrast radiographic studies, CAT scan, and detection of a tumor marker associated with cancer in the blood of the individual. The substance can be administered systemically or locally, usually systemically.

A substance, e.g. a chemotherapeutic drug that reduces cancer cell growth, can be targeted to a cancer cell. Thus, in some embodiments, the invention provides a method of delivering a drug to a cancer cell, comprising administering a complex of drug-polypeptide or drug-polynucleotide to a subject, wherein the complex is specific for a miRNA-regulated polypeptide or the miRNA itself, and the drug is one that reduces cancer cell growth, a variety of which are known in the art and discussed above. Targeting may be accomplished by coupling (e.g., linking, directly or via a linker molecule, either covalently or non-covalently, so as to form a drug-antibody complex) a drug to an antibody specific for a miRNA or a polypeptide regulated by the miRNA. Methods of coupling a drug to form a complex are well known in the art and need not be elaborated upon herein.

Each publication cited in this specification is hereby incorporated by reference in its entirety for all purposes.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention, which will be limited only by the appended claims.

As used herein the singular forms “a”, “and”, and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

### EXPERIMENTAL

### Example 1

### Identification of a Breast Cancer Stem Cell Gene Signature

We previously identified BCSC based on their expression of CD44 and CD24, as being CD44⁺CD24^{-/low}Lineage⁻. Normal breast epithelial cells, defined by the cell surface marker expression, ESA⁺ Lineage⁻ (CD64⁻, CD31⁻, CD140b⁻, CD45⁻), were isolated from three breast reduction samples. By microarray analysis, we looked for differentially expressed genes between BCSC isolated from 6 patients (3 primary malignant pleural effusions and 3 human breast tumors grown as solid tumor xenografts in immunodeficient mice) and normal human breast epithelial cells derived from 3 reduction mammoplasties. A set of 186 genes were selected based on a two-fold difference in expression level with a t-test P value<0.005 across all samples. False discovery rate (FDR) was controlled using the Benjamini-Hochberg procedure. With the above criteria, FDR is less than 5% for the genes in the list. As expected, this cancer stem cell gene signature of 186 genes was sufficient to distinguish breast cancer stem cells from normal breast epithelial cells by gene expression profiling. We also validated the differential expression of these 186 genes by performing real time PCR of 14 randomly selected genes in 3 BCSC samples from xenografts and 1 normal breast epithelium sample. The gene expression patterns seen in individual tumor samples by real time PCR were largely consistent with those observed in the microarray data: in the three tumors tested, we observed consistent expression patterns of all 14 genes, and in the third tumor, expression pattern of 9 out of 14 genes is consistent with the array data. (see Liu, M. F. Clarke, M. F. Association of a Gene Signature from Tumorigenic Breast Cancer Cells with Clinical Outcome, The New England Journal of Medicine, 356: 217-226, 2007, herein specifically incorporated by reference).

BCSCs and non-tumorigenic cancer cells from 10 patient tumors were further screened for the expression of more than 500 miRNAs using an ABI array. Real time RT-PCR was used to confirm these results (Table 2).

Based on the result of microRNA expression, miRNAs were found to play an important role in the regulation of essential BCSC functions. A group of miRNA consisting of miR-182, miR-182, miR-200a, miR-200b, miR-200c are consistently downregulated in breast cancer stem cells. Expression of all 5 of these miRNAs is completely lost in embryonic carcinoma cells (EC cells) but they are expressed in normal embryonic stem cells (ES cells). These data demonstrate the existence of a tumor-initiating cell population with stem cell-like properties in breast cancer, in which miRNA can be used as diagnostic or therapeutic targets.

The targets for these miRNAs were then investigated. m200b and m200c are thought to share the same targets. One target that has been validated for m200b is ZFHX1B, a protein that represses expression of E-cadherin and may play a role in both normal stem cell biology and EMT. Multiple members of the anti-apoptotic proteins in the BCL-2 family are also reported targets. Unregulated expression of BCL-2 family proteins has been implicated in carcinogenesis and the self renewal of normal stem cells.

Four of these mRNAs (m183, m200a, m200b, and m200c) can target BMI1. BMI1 plays a role in the self renewal of both normal stem cells from many tissues and at least some cancer stem cells. Importantly, we find that BMI1 protein is downregulated in the non-tumorigenic cancer cells found in many patients' tumors. The target sequence in the BMI1 mRNA is highly conserved across species, making it likely that it is a bona fide target.

Other interesting targets for these miRNAs are the MYB proto-oncogene, NMYC, IGFBP1, KRAS, FOX01A and Sox2. The MYB and NMYC genes have been associated with normal and malignant stem cell renewal. MYB has recently implicated in breast cancer tumorigenesis. FOX01A plays a role in stem cell maintenance. Several of these genes are overexpressed by the CSCs on the microarrays. Several of these genes are differentially expressed 2-14 fold by the BCSCs as measured by Affymetrix arrays.

### Example 2

Development of markers that can be used as prognostic and predictive tools on formalin fixed paraffin embedded (FFPE) tumor specimen. The sequences identified herein as differentially expressed in BCSC are used to generate markers (in situ hybridization probes) to determine the quantity and location of tumor stem cells in formalin-fixed, paraffin-embedded (FFPE) tissues. All breast cancer biopsies and resection specimens are analyzed by histologic examination which uses thin sections of material that has been embedded in paraffin after fixation in formalin. As such, there exists a very large collection of tumor specimens in the archives of the surgical pathology departments throughout the country that can be used for the histologic study of tumor stem cells and the role that they play in clinical outcome and response to adjuvant therapy.

Tissue microarrays (TMAs) containing Formalin Fixed Paraffin Embedded (FFPE) tumor samples with known clinical outcome are used to determine the clinical significance of these findings. The expression of prognostic or predictive markers by each tumor cell population including normal stromal cells, breast CSCs and the other cancer cells in the tumor is determined.

In situ hybridization probes (ISH) are developed to evaluate gene expression in paraffin embedded tissue. ISH probes are generated in approximately 10 days. These probes have a success rate. The ISH technique is described by St Croix et al and Iacobuzio-Donahue et al. It employs long RNA probes with lengths ranging from 400 to 600 nucleotides and relies on a tyramide based amplification of signal followed by development with either chromogenic or fluorescent substrates. These reagents work very well on paraffin-embedded, formalin-fixed tissue. ISH probes have the advantages over conventional antisera or monoclonal antibodies that one can include sense strands or miss-sense probes as controls. For selected probes, RT-PCR is performed on laser capture dissected material from frozen specimens of breast cancer to verify the expression profile.

The TMAs are built with up to 500 breast carcinomas can be represented in a single TMA block. Breast TMAs include 1) Normal breast tissue microarrays; 2) Annotated breast cancer tissue microarray. Clinical follow-up for these cases will be obtained. 3) To study the variability between breast carcinomas and specifically the degree with which patient-specific factors (as opposed to individual tumor-specific factors) determine the presence of the number of tumor stem cells in the individual cancer specimens a TMA is generated with breast carcinoma material from patients with 2 independent breast cancer primaries. 4) To study the effect of the metastasis process on a number of tumor stem cells in breast cancer a tissue microarray is generated in which material from 20 patients is represented. For each patient the primary breast tumor is represented together with one or more lymph node metastasis and a metastasis from a distant site such as brain, lung, or bone. 5) A breast cancer tissue microarray containing specimens from primary invasive breast cancer, in which outcome data are available for all patients, with median follow-up of 15.4 years (range 6.3-26.6 years), may also be used. The follow-up includes overall survival, disease-specific survival and time to first recurrence.

Determining presence of miRNA species in histologic sections. miRNA species are useful as markers for tumor stem cells. In performing in situ hybridization for miRNA species locked nucleic acid (LNA) is used, which has a much higher melting temperature than RNA. Using LNA probes tissue microarrays are examined with known positive and negative controls for each miRNA species (as proven by RT-PCR) and methodically vary a wide range of melting temperatures in experiments. Negative controls consisting of mismatched LNA probes with 1, 2, 3 or 4 mismatches are used.

A second approach to determine the presence of miRNA species in various components (tumor cells versus stromal cells) combines RT-PCR with laser capture microdissection. Using as few as 25 cells, enough material can be generated after linear amplification to determine the quantity of −500 different miRNA species with confidence. This number of cells is easily obtainable through laser capture microdissection. Analyses of these breast cancer TMAs with the miRNA markers enables determination in a retrospective manner of the best probes.

### Example 3

Target pathways that render CSCs resistant to standard cytotoxic chemotherapies. Exogenous miRNAs or synthetic shRNAs are used to target pathways that make CSCs resistant to treatment. Three different published methods are used to deliver the shRNA: liposomal delivery (see Sorensen et al. (2003) J Mol Biol 327, 761-6), conjugation of the shRNA with atellocolagen (see Takeshita et al. (2005) Proc Natl Acad Sci USA 102, 12177-82), and conjugation of the shRNA with a monoclonal antibody/protamine complex (see Song et al. (2005) Nat Biotechnol 23, 709-17). The third method utilizes an antibody that can specifically target the cancer cells. In the latter case, antibodies that specifically bind to CSCs or antibodies that target all of the cancer cells are tested. Flow cytometry is used to identify the antibodies that will target the CSCs or all of the cancer cells in a particular xenograft tumor.

Xenograft tumors established from 6 different patients' tumors are tested to determine whether systemic delivery of the shRNA augments chemotherapy (cytoxan, taxol and adriamycin) or radiation therapy. Xenograft tumors are established, and when they reach a size of 0.5 cm the mice are treated with one of the cytotoxic agents and either the experimental or control shRNA delivered by liposomes, conjugation with atellocolagen, or one of the monoclonal antibody/protamine complexes. Tumor volume is followed for 4 months post treatment. Each experimental group contains at least 10 mice, and experiments are repeated 3 times. In addition, tumors from 5 mice treated with either the control or experimental shRNA virus are removed and analyzed to make certain that the shRNA downregulates the protein of interest in vivo.

shRNAs that target pathways such as BMI1, MYB, PTEN, STAT, miRNAs differentially expressed by CSCs and other pathways are delivered to determine the effect on the survival and self renewal of breast cancer stem cells. These experiments are performed as described above. For example, a miRNA that is underexpressed in CSCs is systemically delivered to determine therapeutic potential.

### Example 4

### Down-Regulation of MicroRNA Clusters Links Normal and Malignant Breast Stem Cells

Human breast cancers contain an apparent cancer stem cell population (BCSCs) with properties reminiscent of normal adult and embryonic stem cells. Molecular regulators of self renewal and differentiation shared by normal and malignant stem cells have yet to be described. We found that 37 miRNAs (miRNAs) were differentially expressed by BCSCs and non-tumorigenic cancer cells. Three clusters, miR-200c-141, miR-200b-200a-429 and miR-183-96-182 were downregulated in normal breast stem cells, in human breast cancer stem cells and in embryonal carcinoma cells. Expression of SOX2, a known regulator of embryonal stem cell self-renewal and differentiation, was modulated by miR-200c. In addition, expression of miR-200c and miR-183 suppressed the growth of embryonal carcinoma cells in vitro, abolished their tumor-forming ability in vivo, and inhibited the clonogenicity of breast cancer cells in vitro. The down-regulation of these 3 miRNA clusters provides a molecular link that connects breast cancer stem cells and normal stem cell biology.

In this study, we identified 3 clusters of miRNAs that were specifically down-regulated in normal murine breast stem cells, human breast cancer stem cells and human embryonal carcinoma cells. Expression of miR-200c and miR-183, miRNAs which are located in 2 of the down-regulated clusters, suppressed growth of embryonal carcinoma cells in vitro, inhibited their tumorigenicity in vivo and strongly repressed clonogenicity of breast cancer cells by impairing stem/progenitor cell maintenance. Our results indicate that down-regulation of the 3 miRNA clusters regulates stem cell self-renewal pathways in both normal and malignant stem cells.

### Results

mRNA Profiling of Human Breast and Embryonal Cancer Cells. As miRNAs are critical regulators involved in self-renewal and differentiation of normal embryonic and tissue stem cells, we compared the miRNA expression profile between human CD44⁺CD24^{-/low} lineage⁻ breast cancer cells (TG cells) and the remaining lineage⁻ non-tumorigenic breast cancer cells (NTG cells). In many patients with breast cancer, a minority population of CD44⁺CD24^{-/low} lineage⁻ cancer cells is highly tumorigenic in immunodeficient mice, in comparison to the remaining lineage⁻ breast cancer cells. The CD44⁺CD24^{-/low} lineage⁻ cells have stem cell like properties such as self-renewal and differentiation, and can regenerate the original tumor from as few as 200 cells, whereas tens of thousands of the remaining lineage⁻ non-tumorigenic cancer cells can not.

Multiplex real-time PCR was used to measure the expression of 460 miRNAs in TG cells and NTG cells isolated from three human breast tumors. We found that 37 miRNAs were up-regulated or down-regulated in TG cells compared to NTG cells in all three samples analyzed (FIG. 1A). The expression of these 37 differentially expressed miRNAs was then measured in a total of 11 sets of human TG cells and NTG cells, and this analysis confirmed that these 37 miRNAs were indeed differentially expressed (FIG. 1B). Three clusters of miRNAs, the miRNA-200c-141 cluster located on chromosome 12p13, the miR-200b-200a-429 cluster located on chromosome 1p36, and the miR-183-96-182 cluster located on chromosome 7q32, were consistently down-regulated in human breast cancer TG cells (FIG. 1C). For example, expression of miR-200a, miR-200b, and miR-200c was 2 to 218 times lower in TG cells compared to NTG cells.

It is thought that the CD44⁺CD24^{-/low} lineage⁻ cells are malignant counterparts of normal mammary stem or early progenitor cells. Similarly, embryonic carcinoma cells are malignant cells that arise from germ cells, which share many properties with pluripotent stem cells. Thus, the expression of these miRNAs was tested in Tera-2 embryonal carcinoma cells. Notably, Tera-2 cells either fail to express detectable levels of each of the miRNAs, or the level of expression is just at the level of detection (FIG. 1D). When expression levels were compared to breast cancer cells, Tera-2 cells expressed at least 4-fold less of all of these miRNAs than breast cancer NTG cells did. The miRNA seed sequence serves to direct the miRNA to its mRNA targets. Remarkably, the miR-200c-141 cluster and the miR-200b-200a-429 cluster are formed by two groups of miRNAs with essentially the same seed sequence (miR-200c/miR-200b/miR-429 miRNAs, and miR-200a/miR-141 miRNAs) (FIG. 1C). Given this similarity and the observed expression patterns, down-regulation of all 3 of the clustered miRNAs in breast cancer CD44⁺CD24^{-/low} lineage⁻ cells and Tera-2 embryonal carcinoma cells is critical to maintain a stem cell function in cancer cells.

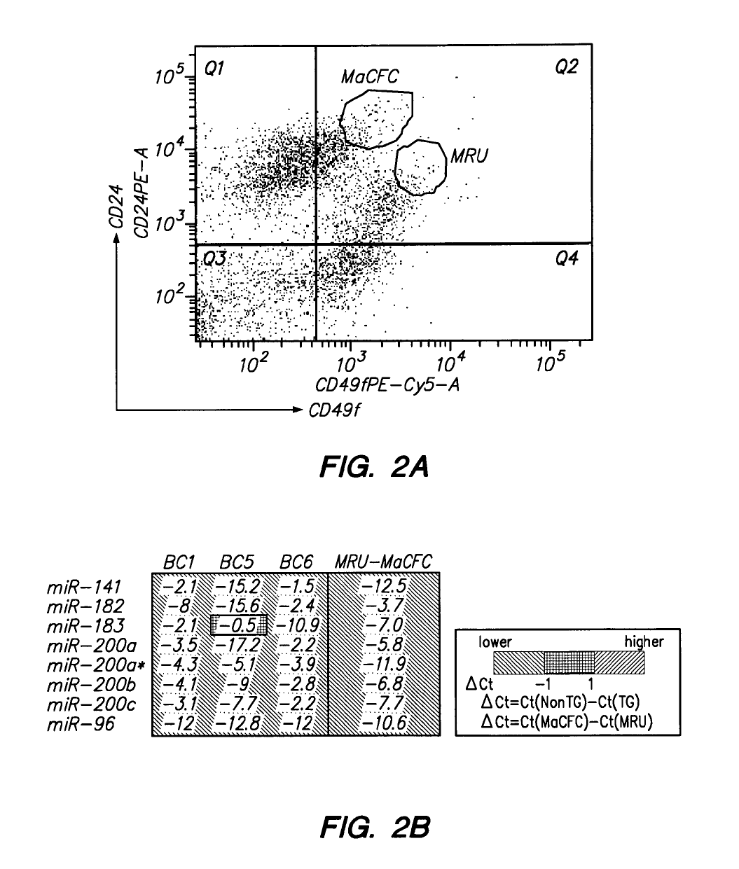

mRNA Expression Connects Normal Mammary Development and Breast Cancer Stem Cell Differentiation. The functional similarities of cancer cells with normal tissue stem cells suggest that activation of normal stem cell self-renewal and/or differentiation pathways account for many of the properties associated with malignancies. We therefore tested early mammary stem and progenitor cells and more differentiated mammary epithelial progenitor cells for the expression of the miRNAs that are differentially expressed by breast cancer TG cells and NTG cells. Although the cellular hierarchy of the mouse mammary epithelium is still only partially understood, CD24^{med}CD49f^{high}CD29^{high}Sca-1⁻ mouse mammary fat pad cells are enriched for mammary stem cells with an ability to regenerate a whole mammary gland in vivo. We collected the CD24^{med}CD49f^{high}CD45CD31⁻CD140a⁻Ter119⁻ cells (MRUs) that are enriched for mammary stem cells and the CD24^{high}CD49f^{low}CD45⁻CD31⁻CD140a⁻Ter119⁻ cells (MaCFCs) that are enriched for more differentiated mammary epithelial progenitor cells (FIG. 2A). We found that all three of the clustered miRNAs that were down-regulated in human breast cancer TG cells were also down-regulated in mouse MRU cells as compared to MaCFCs (FIG. 2B). This demonstrates that the differential expression of these 3 miRNA clusters between breast cancer TG cells and NTG cells is a key component of a normal mammary cell developmental pathway.

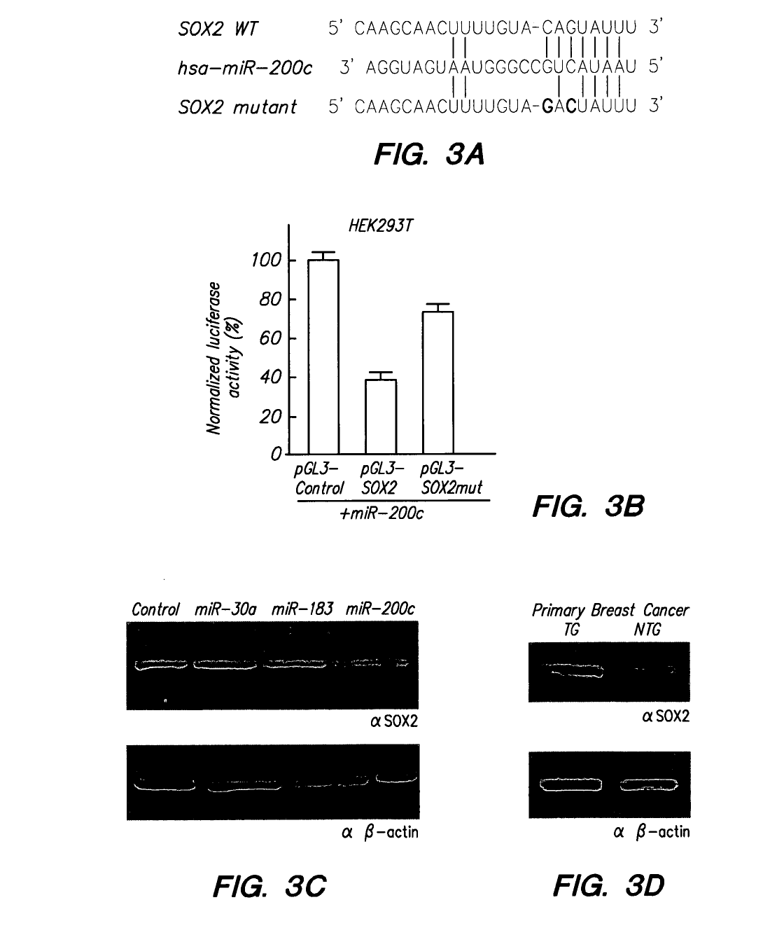

MiR-200c Targets SOX2. Potential molecular targets of miR-200bc/429 were predicted by TargetScan 4.2. Among the potential targets, we focused on SOX2 because it possessed critically conserved nucleotides indicative of a legitimate target and is known to be essential in regulating self-renewal and differentiation of other stem cell types, including embryonic stem cells. The ability of miR-200c to regulate the 3′UTR of SOX2 was evaluated via luciferase reporter assays. HEK293T cells, which did not express miR-200c and miR-429 and expressed barely detectable levels of miR-200b were used. The 3′UTR target sites of SOX2 were cloned into pGL3-Control vector, downstream of a luciferase minigene. HEK293T cells were co-transfected with a pGL3 luciferase vector, pRL-TK Renilla luciferase vector and miR-200c precursor RNA. We observed that the luciferase activity was suppressed by 60% for SOX2 (FIG. 3B); moreover, mutation of the miRNA-200bc/429 seed region abrogated the ability of the miRNA to repress expression of SOX2, demonstrating specificity of the target sequence for SOX2 (FIGS. 3A and 3B).

The ability of miR-200c to regulate endogenous SOX2 protein was also tested. To do this, we infected Tera-2 cells with a lentivirus that expressed miR-200c. Infected cells were collected by flow cytometry. Western blotting showed that SOX2 protein expression was decreased in cells that expressed miR-200c (FIG. 3C). In contrast, the negative controls, miR-30a and miR-183, did not modulate SOX2 protein expression. Then we examined SOX2 expression in tumorigenic CD44⁺CD24^{-/low} lineage⁻ cells (TG cells) and NTG cells collected from a primary human breast cancer sample. As shown in FIG. 3D, SOX2 protein expression was clearly lower in breast cancer NTG cells as compared to TG cells.

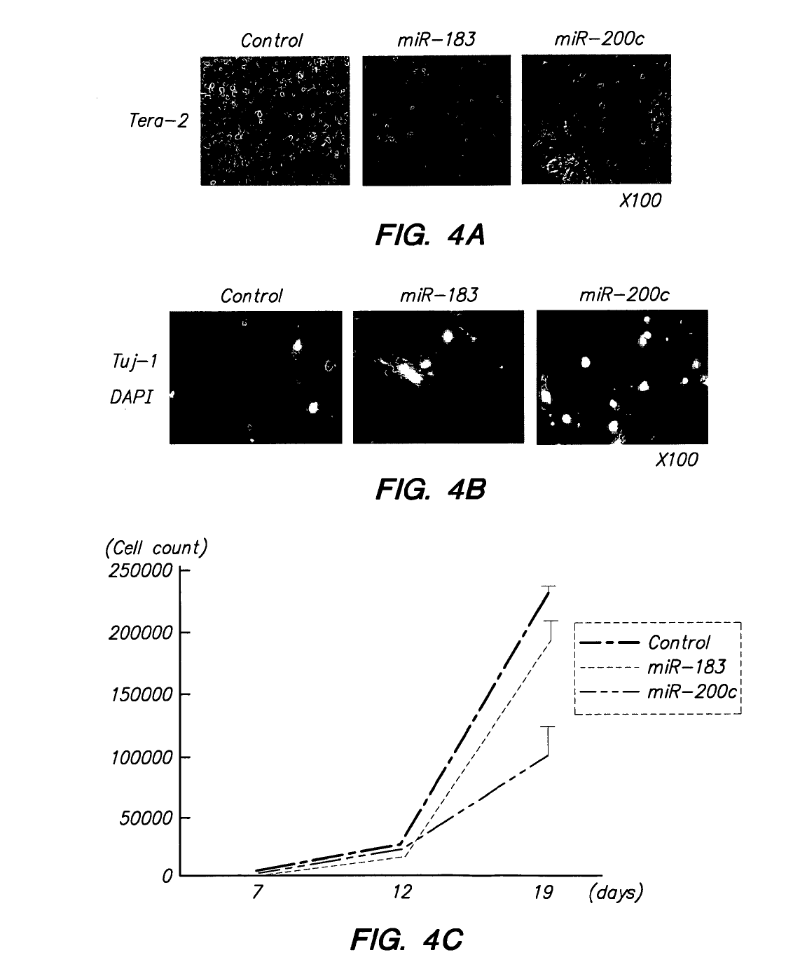

MiR-200c and miR-183 Suppress Cancer Cell Growth in vitro. The observation that the same clusters of miRNAs were down-regulated in normal mammary stem cells, tumorigenic CD44⁺CD24^{-/low} lineage⁻ breast cancer cells and embryonal cancer cells implies that these miRNAs are regulators of critical stem cell functions such as self-renewal and/or differentiation. Indeed, it has recently been shown that miR-200 family miRNAs prevent EMT (epithelial to mesenchymal transition) by suppressing expression of ZEB1 and ZEB2, transcriptional repressors of E-cadherin. EMT is a stem cell property that has been linked to both normal and cancer stem cells. To determine how expression of some of these miRNAs affects cells, we infected cells with lentivirus vectors that express miR-200c or miR-183. The morphology of the cells infected with either the miR-200c or miR-183 lentiviruses suggested that they had differentiated (FIG. 4A). Indeed, staining with anti-neuron specific class 1116 tubulin (Tuj-1) antibody showed that miR-200c infected Tera-2 cells preferentially expressed the early post-mitotic neuron marker, Tuj1 antigen, suggesting that the miRNAs had induced neural differentiation (FIG. 4B).

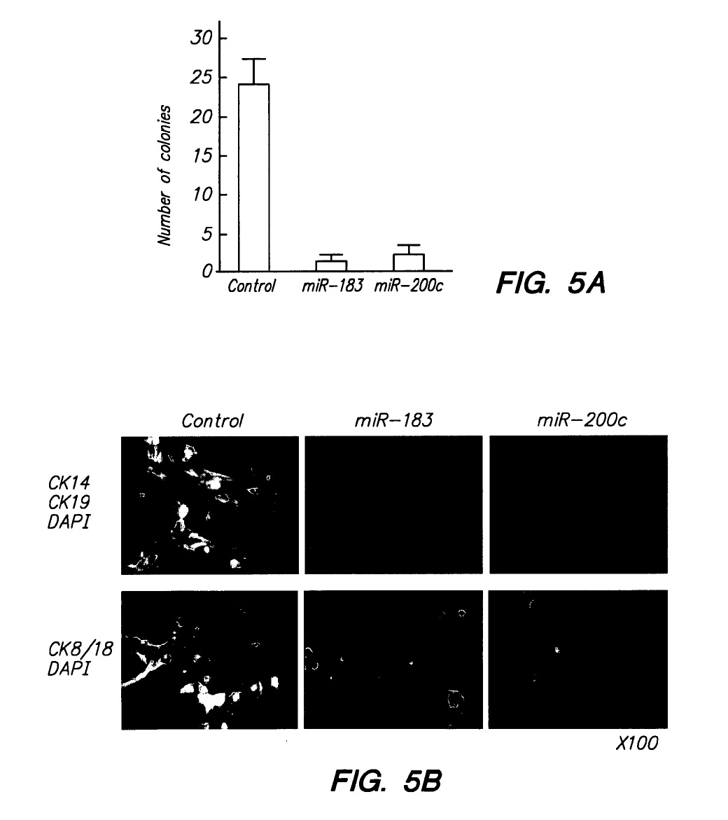

We found that Tera-2 cells infected with either the miR-200c or the miR-183 lentivirus, but not the control lentivirus, showed growth retardation (FIG. 4C). Growth retardation by miR-200c was stronger than miR-183 reflecting the strength of neural differentiation observed (FIGS. 4B and 4C). The mouse MMTV-Wnt-1 murine breast tumor is composed of both luminal and myoepithelial cells and an expanded mammary stem cell pool. We infected MMTV-Wnt-1 murine breast cancer cells with a miR-200c or a miR-183 expressing lentivirus. Colony formation by the miR-183 or miR-200c infected cells was almost completely suppressed, reducing the number of colonies by 96% for miR-200c and by 94% for miR-183 when compared to cells infected with the control lentivirus (FIG. 5A).

Normal breast stem/progenitor cells (MRUs, mammary repopulating units) and MMTV-Wnt-1 breast cancer stem cells are bi-phenotypic expressing both the myoepithelial cell cytokeratin CK14 and the epithelial cell cytokeratin CK8/18. Mature epithelial cells express either CK8/18 or CK19 but not CK14. Myoepithelial cells express CK14 but not CK8/18 or CK19. Breast cancer cells infected with control virus formed large colonies and expressed CK14 and CK8/18, with an occasional cell that expressed CK19 (FIG. 5B), whereas cells infected with the miR-183 or miR-200c expressing virus formed only small aggregates of cells that showed low levels of CK14 (FIG. 5B). These results show that miR-183 and miR-200c infected breast cancer cells have lost the progenitor phenotype and expression of miR-183 and miR-200c induced the differentiation of breast cancer stem cells in vitro.

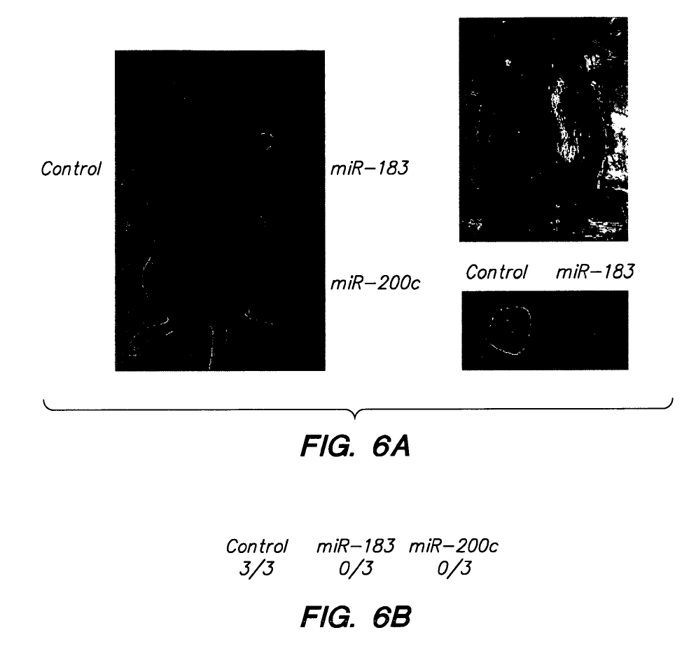

Suppression of Tumorigenicity of Embryonic Carcinoma Cells by miR-200c and miR-183. In order to determine the significance of the effect of miR-200c and miR-183 on the growth of cancer cells in vivo, Tera-2 embryonal carcinoma cells were infected with the lentivirus expressing miR-200c or miR-183, or a control lentivirus, and the infected cells were collected by flow cytometry. Then the infected Tera-2 cells were injected subcutaneously into immunodeficient NOD/SCID mice. Remarkably, two months later, we observed that 50,000 Tera-2 cells infected with control lentivirus formed a tumor in 3/3 mice injected, whereas 0/3 mice receiving the miR-200c infected and 0/3 miR-183 infected Tera-2 cells had tumors (FIG. 6).

The results reported here demonstrated that miR-200c-141, miR-200b-200a-429 and miR-183-96-182 were down-regulated in normal breast stem cells, in human breast cancer stem cells and in embryonal carcinoma cells and that expression of SOX2 was modulated by miR-200c. SOX2 is a member of the HMG-domain protein family and forms a complex with OCT4 to bind DNA, regulate transcription and direct the processes of self-renewal and differentiation in embryonic stem cells, and some lineage specific stem cells such as neural stem cells. Here, we observed that expression of miR-200c and miR-183 also suppressed the growth of embryonal carcinoma cells in vitro, induced their neural differentiation, abolished their tumor-forming ability in vivo, and inhibited the clonogenicity of breast cancer cells in vitro. Thus our data on differential miRNA expression provide a molecular link between cancer stem cells and embryonic stem cells, as well as a molecular explanation for the increased tumorigenicity displayed by the subpopulation of CD44⁺CD24^{-/low} lineage⁻ breast cancer cells in many patients' tumors.

Our analysis revealed 5 differentially-expressed miRNAs that were down-regulated, shared the same seed sequences, and yet mapped to two clusters on different chromosomes. The functional redundancy of these families of miRNAs may reflect a failsafe mechanism, to maintain stem cell homeostasis and prevent tumors, by ensuring that a single mutation does not perturb the regulation of their targets. The regulation of SOX2 by the miRNAs is intriguing. Indeed, SOX2 along with OCT4, is essential not only for ESC self-renewal and maintenance of pluripotency, but also is a core factor in reprogramming of somatic cells to induced pluripotent stem cells (iPSCs). Considering these observations, along with the reported link of SOX2 to breast cancer, a shared and extensive regulatory gene network underlies both cancer stem cell and embryonic stem cell self-renewal and differentiation.

The miRNA profile described in these studies undoubtedly points to many other factors that likely link regulation of vital cancer and normal stem cell functions. Prediction programs such as Targetscan4.2 suggest that there are likely many other genes functionally important for stem cells that are regulated by miRNA-200c-141, miR-200b-200a-429, and miR-183-96-182.

Other miRNAs identified in our screen also are likely to be important for tumorigenicity. For example, our data analysis also indicated that miR-155 is highly expressed in the breast cancer stem cells relative to the other cancer cells. Notably, miR-155 was originally identified as the product of the oncogenic BIC gene locus in B cell lymphoma and high levels of expression are associated with poor prognosis of lung adenocarcinoma patients. Abnormal proliferation and myelodysplasia is seen when miR155 expression is sustained in the blood system. Thus, increased expression of miR-155 is also a hallmark of breast cancer stem cells that may signify increased proliferation of these cells relative to their non-tumorigenic counterparts.

EMT is a widespread, developmental program that regulates cell migration in many tissues and organs, and is associated with normal and malignant mammary stem cell function. Recent studies have shown that expression of components of the EMT pathway including SNAI2 is highest in the CD44⁺CD24^{-/low} lineage⁻ breast cancer cells. Here we show that that miR-200 family miRNAs were strongly suppressed in human breast tumorigenic CD44⁺CD24^{-/low} lineage⁻ cells. The miR-200 family of miRNAs suppresses the translation of ZEB1 and ZEB2 that serve as EMT inducers. Multiple sites in the 3′UTR of ZEB1 and ZEB2 are targeted by the miR-200 family miRNAs, with suppression of ZEB1 and ZEB2 up-regulating expression of E-cadherin and inhibiting EMT. Collectively these findings demonstrate the miR-200 family miRNAs as important regulators of stem cell function by controlling the EMT process in both normal and malignant breast stem cells.

In summary, the present findings provide a strong molecular link between normal breast stem/progenitor cells, the CD44⁺CD24^{-/low} lineage⁻ breast cancer cells and embryonal carcinoma cells. The facts that the miR-200a, miR-200b, miR-200c and miR-141 are significantly down-regulated both in a subset of human breast cancer cells and in normal mammary stem cells and that miR-200c regulates self-renewal gene, suggest that normal stem cells and CD44⁺CD24^{-/low} lineage⁻ breast cancer cells share common molecular mechanisms that regulates stem cell functions such as self-renewal and EMT.

### Experimental Procedures

Cell Culture. Human embryonal kidney (HEK) 293T cells were maintained in Dulbecco's modified Eagle's medium (DMEM) with 10% FBS, 100 U/mL penicillin, 100 μg/mL streptomycin, and 250 ng/mL amphotericin B (Invitrogen) and incubated at 5% CO₂ at 37° C. The human embryonal carcinoma cell line Tera-2 (HTB-106) was purchased from ATCC, and grown in modified McCoy's medium (Invitrogen) with 100 units/ml of penicillin G, 100 μg/ml of streptomycin, and 250 ng/ml of amphotericin B supplemented with 15% fetal bovine serum and incubated at 5% CO₂ at 37° C.

Preparation of Single Cell Suspensions and Flow Cytometry. Primary Breast Cancer specimens were obtained from the consented patients as approved by the Research Ethics Boards at Stanford University, and the City of Hope Cancer Center in California. Tumor specimens were mechanically dissociated and incubated with 200 U/ml Liberase Blendzyme 2 (Roche). Cell staining and flow cytometry was performed as described previously. Mouse normal breast specimens were mechanically dissociated and incubated with 200 U/ml Liberase Blendzyme 4 (Roche). Cell staining and flow cytometry was performed as described previously.

Transplantation of Embryonal Carcinoma Cells into NOD/SCID Mice. NOD/SCID mice (Jackson laboratory) were anesthetized using 1-3% isoflurane. Embryonal carcinoma cells were suspended in Matrigel (BD Biosciences) and injected subcutaneously into NOD/SCID mice. All experiments were carried out under the approval of the Administrative Panel on Laboratory Animal Care of Stanford University.

Multiplex real-time PCR assay. Eleven sets of CD44⁺CD24^{-/low} lineage⁻ tumorigenic and the remaining lineage non-tumorigenic human breast cancer cells were isolated using a BD FACSAria sorter as previously described). mRNA profiling was performed by multiplex real-time PCR. For RNA preparation, 100 tumorigenic CD44⁺CD24^{-/low} lineage⁻ human breast cancer cells and the other non-tumorigenic lineage⁻ cancer cells were double-sorted into Trizol (Invitrogen) and RNA was extracted following the manufacturer's protocol. Glycogen (Invitrogen) was used as a carrier for precipitation. RT, pre-PCR and the multiplex real-time PCR were performed as described previously. Briefly multiplex reverse transcription reactions were performed with 466 sets of second strand synthesis primers. Then multiplex pre-PCR reactions were performed with 466 sets of forward primers and universal reverse primers. The multiplex pre-PCR product was diluted 8 times, aliquoted into 384 well reaction plates and the abundance of each miRNA was measured individually. Each primer and probe contained zip-coded sequences specifically assigned to each miRNA to increase the specificity of each reaction, so that even small sequence differences in miRNA were amplified and detected. This approach is specific for detection of mature miRNAs and reliable as miRNA measurements on RT-PCR and microarray are concordant. Results were normalized by the amount of small nuclear RNA expression, C/D box 96A and C/D box84. The difference of miRNA expression between two populations were calculated as; ΔCt=normalized Ct (tumorigenic cells)-normalized Ct (non-tumorigenic cells).

Plasmid Vectors and Mutagenesis. The multiple cloning site of pGEM-T-Easy vector (Promega) was amplified by PCR and was inserted into the pGL3 control vector (Promega) at the XbaI site (pGL3-MC). A 553 by fragment of the SOX2 3′UTR (corresponding to positions of 1620-2172 of the NM_{—}003106.2) was amplified by PCR using the cDNA of HEK293T cells as a template, and cloned into the pGEM-T-Easy vector. The SOX2 3′UTR product was cloned at the 3′ of the luciferase gene of pGL3-MC vector. All products were sequenced. Mutations of the putative miR-200c target sequence within the 3′UTR of SOX2 were generated using QuikChange Site-Directed Mutagenesis kit (Stratagene).

Luciferase Reporter Analysis. HEK293T cells were seeded at 1×10⁵ cells per well in 48-well plates the day prior to transfection. All transfections were carried out with Lipofectamine 2000 (Invitrogen), according to the manufacture's instructions. Cells were transfected with 320 ng pGL3 luciferase expression construct containing the 3′UTR of human SOX2, 40 ng pRL-TK Renilla luciferase vector (Promega), and 50 nM hsa-miR-200c precursor (Ambion). 48 h after transfection cells were lysed and luciferase activities were measured using the Dual-Luciferase Reporter Assay System (Promega) and normalized to Renilla luciferase activity. All experiments were performed in duplicate with data pooled from three independent experiments.

Lentivirus Production. The sequences of miR-200c and miR-183 including stem loop structure and 200-300 base pairs of up-stream and down-stream flanking genomic sequence were cloned by PCR using cDNA of HEK293T or MCF7 cells as a template. The products are cloned into HpaI and XhoI sites of pLentiLox 3.7 vector. To produce the control vector, the U6 promoter sequence was removed by XbaI and HpaI digestion, incubated with Klenow enzyme and ligated. Lentiviruses were produced as described (Tiscornia et al. (2006). Nat Protoc 1, 241-245).

Western Blotting. Tera-2 cells were infected by lentiviruses expressing miRNA and infected cells were collected by flow cytometry. Human breast cancer cells were collected by flow cytometry as described above. The collected cells were lysed in SDS sample buffer (50 mM Tris-HCl pH 6.8, 2% SDS, 10% glycerol 5 mM EDTA 0.02% Bromophenol Blue, 3% β-mercaptoethanol). Samples were separated on SDS-8% polyacrylamide gel electrophoresis and transferred to polyvinylidene difluoride filters (Amersham). After blocking with 5% skim milk in 0.05% Tween 20/PBS, filters were incubated with 1:2000 (1:1000 for primary breast cancer samples) diluted anti-SOX2 polyclonal antibody (Millipore) or 1:2000 diluted anti-β-actin antibody (Santa Cruz Biotech). Then 1:10,000 diluted peroxidase-conjugated donkey anti-rabbit or sheep anti-mouse IgG antibody (Amersham) was added and developed using the Western Blotting Luminol Reagent (Santa Cruz Biotech).

Breast Cancer Cell Colony Formation Assay. Mouse MMTV-Wnt1 tumors were digested using 200 U/ml Liberase Blendzyme 2 (Roche) and dissociated as described (Cho et al., 2008 Stem Cells 26, 364-371). Cells were stained with anti-CD31, CD45, and CD140a antibodies and lineage positive cells were depleted by flow cytometry. 15,000 cells were infected with 20 MOI of miRNA expressing lentiviruses by spin infection for 2 hours followed by incubation at 37° C. for 2 hours in DMEM/F12 supplemented with 5% BSA, 2% heat inactivated FBS, 1:50 B27, 20 ng/mL EGF, 20 ng/mL bFGF, 10 μg/mL insulin, and 10 μg/mL heparin. The infected cells were washed twice with the same medium and then the medium was replaced by Epicult medium (Stemcell technologies) with 5% FBS. The infected cells were plated on the 30,000 irradiated 3T3 feeder cells in the 24-well plate. The medium was replaced again by Epicult medium without serum 24 hours after seeding and cells were incubated for 6 days at 5% CO₂ at 37° C.

Immunofluorescence. Tera-2 cells were infected by lentiviruses expressing miRNA and infected cells were collected by flow cytometry. 1×10⁴ cells were grown in a well of 24-well plate and washed twice with PBS (20 mM potassium phosphate pH 7.4, 150 mM NaCl). Cells were fixed with methanol/acetone (1:1), washed twice with 0.1% Tween 20/PBS, and incubated in 1% Triton X/PBS for 30 min. Cells were blocked with 4% goat serum in PBS and incubated with primary antibody (1:750 dilution for anti-Tuj1 monoclonal antibody (Covance)), again washed three times in 0.1% Tween 20/PBS, and then stained with 1:300 diluted Alexa Fluor 488-conjugated anti-mouse IgG antibody (Invitrogen). Breast cancer cells were stained by using the fixation solutions with BrDU Flow Kits (BD Pharmingen). Cells were blocked with 4% goat serum in PBS and incubated with primary antibody (1:200 dilution for rabbit anti-cytokeratin 14 (Covance), rat anti-cytokeratin 19, and rat anti-cytokeratin 8/18 antibodies (Developmental Studies Hybridoma Bank, DSHB)), again washed three times in 0.1% Tween 20/PBS, and then stained with 1:200 diluted Alexa Fluor 488-conjugated anti-rat IgG antibody and 1:200 diluted Alexa Fluor 594-conjugated anti-rabbit IgG antibody (Invitrogen). The stained cells were observed using a fluorescent microscope (Leica DMI 6000 B).

### Example 5

### miR-200 Suppresses Normal Mammary Outgrowth

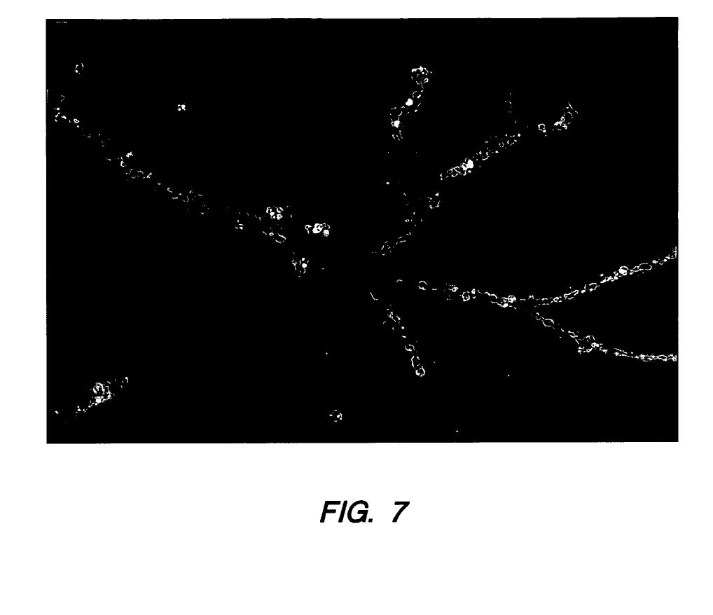

As shown in FIG. 7, miR-200 suppresses normal mammary stem cells. 50,000 normal mouse mammary cells were infected by miR-200c expressing lentivirus or control lentivirus and injected into cleared mammary fat pad of the weaning age mice. Growth of the GFP-expressing mammary tree was analyzed 6 weeks after injection. FIG. 7 illustrates the GFP-expressing mammary tree formed by control lentivirus infected mammary cells, shown by 2/5 branch formations. In contrast, where the GFP was expressed, indicating expression of miR-200c, there were 0/5 branches formed.

It was also found that miR-200c and miR-183 suppress human breast cancer growth. 10,000 tumorigenic cancer (TG) cells were isolated from human breast xenograft tumor, and infected by miRNA-expressing or control lentivirus and injected into mammary fat pads of the NOD/SCID mice. Tumor incidence was analyzed 16 weeks after injection. In the control animals, there were tumors in 4/5 animals, while in the cells expressing miR-200c there were 1/5 tumors, and in the cells expressing miR-183 there were 0/2.

What is claimed is:

1. A method for identifying cancer stem cells comprising:
contacting a sample with reagents specific for at least one miRNA selected from miR-214; miR-127; miR-142-3p; miR-199a; miR-409-3p; miR-125b; miR-146b; miR-199b; miR-222; miR-299-5p; miR-132; miR-221; miR-31; miR-432; miR-495; miR-150; miR-155; miR-338; miR-34b; miR-212; miR-146a; miR-126; miR-223; miR-130b; miR-196b; miR-521; miR-429; miR-193b; miR-183; miR-96; miR-200a; miR-200c; miR-141; miR-182; miR-200a; miR-200b,
wherein cancer stem cells express altered levels of the said at least one miRNA relative to non-tumorigenic cells.

2. The method according to claim 1, wherein quantifying of miRNA expression is performed by in situ hybridization.

3. The method according to claim 1, where in quantifying is performed by real-time polymerase chain reaction.

4. The method according to claim 1, wherein said patient is human.

5. The method according to claim 4, where in said human is undergoing cancer treatment.

6. The method according to claim 1, further comprising contacting a sample with reagents specific for proteins regulated by said miRNAs,
wherein cancer stem cells express altered levels of the said proteins relative to non-tumorigenic cells.

7. A method of screening a candidate chemotherapeutic agent for effectiveness against a CSC, the method comprising:
contacting said agent with the CSC, and
determining the effectiveness of said agent in altering intracellular levels of at least one miRNA selected from miR-214; miR-127; miR-142-3p; miR-199a; miR-409-3p; miR-125b; miR-146b; miR-199b; miR-222; miR-299-5p; miR-132; miR-221; miR-31; miR-432; miR-495; miR-150; miR-155; miR-338; miR-34b; miR-212; miR-146a; miR-126; miR-223; miR-130b; miR-196b; miR-521; miR-429; miR-193b; miR-183; miR-96; miR-200a; miR-200c; miR-141; miR-182; miR-200a; miR-200b.

8. A method of altering tumorigenicity in a cancer stem cell, the method comprising:
altering the activity of a microRNA expressed in said cell, selected from miR-214; miR-127; miR-142-3p; miR-199a; miR-409-3p; miR-125b; miR-146b; miR-199b; miR-222; miR-299-5p; miR-132; miR-221; miR-31; miR-432; miR-495; miR-150; miR-155; miR-338; miR-34b; miR-212; miR-146a; miR-126; miR-223; miR-130b; miR-196b; miR-521; miR-429; miR-193b; miR-183; miR-96; miR-200a; miR-200c; miR-141; miR-182; miR-200a; and miR-200b.

9. The method according to claim 8, wherein said microRNA is selected from miR-200c, miR-141, miR-200b, miR-200a, miR-429, miR-182, miR-96, and miR-183 and wherein the method comprises upregulating activity.

10. The method according to claim 9, wherein said agent comprises a miRNA genetic sequence selected from miR-200c, miR-141, miR-200b, miR-200a, miR-429, miR-182, miR-96, and miR-183, and operably linked to a promoter active in said cell.

11. The method according to claim 10, wherein said altering step is performed in vitro.

12. The method according to claim 10, wherein said altering step is performed in vivo.

13. The method according to claim 10, wherein the cancer stem cell is a breast cancer stem cell.

14. The method of claim 13, wherein the breast cancer stem cell is CD44⁺CD24^{-/low} lineage⁻.

15. The method according to claim 8, wherein said altering step comprises administering to said cell an agent that decreases the level of said miRNA in said cell.

16. The method according to claim 15, wherein said agent is an anti-sense oligonucleotide.
